(19) United States
(12) Patent Application Publication
(10) Pub. No.: US 2026/0085288 A1
(43) Pub. Date: Mar. 26, 2026

(54) METHOD OF DIFFERENTIATION OF PLURIPOTENT STEM CELLS TO HEMATOPOIETIC PRECURSOR AND STEM CELLS

(71) Applicant: R.P. SCHERER TECHNOLOGIES, LLC, Carson City, NV (US)

(72) Inventors: Boris GREBER, Dusseldorf (DE); Daniel TERHEYDEN-KEIGHLEY, Dusseldorf (DE)

(21) Appl. No.: 19/111,288

(22) PCT Filed: Sep. 12, 2023
(86) PCT No.: PCT/US2023/032541

§ 371 Date: Mar. 12, 2025

Related U.S. Application Data
(60) Provisional application No. 63/406,185, filed on Sep. 13, 2022.
(60) Provisional application No. 63/449,506, filed on Mar. 2, 2023.

(51) Int. Cl. C12N 5/0783; C12N 5/0786; C12N 5/0789

(52) U.S. Cl. CPC C12N 5/0646; C12N 5/0636; C12N 5/0645; C12N 5/0647; C12N 2501/125; C12N 2501/155; C12N 2501/165; C12N 2501/2307; C12N 2501/2315; C12N 2501/26; C12N 2501/415; C12N 2506/02; C12N 2506/45; C12N 2533/52

(57) ABSTRACT

The invention provides a method of producing a population of CD34⁺ hematopoietic precursor cells. The CD34⁺ hematopoietic precursor cells are used in methods of producing natural killer (NK), methods of inducing NK cell differentiation from pluripotent stem cells (PSCs), and methods of generating terminally differentiated hematopoietic cells from PSCs. The differentiation of immune cells such as NK cells from PSCs includes the use of a hemogenic endothelium induction cocktail that includes a WNT signaling pathway activator, a bone morphogenetic protein and/or a vascular endothelial growth factor. Also provided is a method of producing hematopoietic stem cells from pluripotent stem cells.

### CROSS-REFERENCE TO RELATED APPLICATIONS

This application claims benefit of priority under 35 U.S.C. § 119(e) of U.S. Provisional Application No. 63/406,185, filed Sep. 13, 2022, and U.S. Provisional Application No. 63/449,506, filed Mar. 2, 2023. The disclosures of the prior applications are considered part of and are herein incorporated by reference in the disclosure of this application in their entirety.

### BACKGROUND OF THE INVENTION

### Field of the Invention

The present invention relates generally to hematopoietic cells and more specifically to methods of generating hematopoietic precursor and stem cells, from pluripotent stem cells (PSCs).

### Background Information

Pluripotent stem cells are cells that are capable to self-renew and to give rise to all cells of the three primary groups of cells that make up a human body, including: ectoderm (skin and nervous system cells), endoderm (including gastrointestinal and respiratory tracts cells, endocrine glands cells, liver cells, and pancreas cells), and mesoderm (including bone, cartilage, most of the circulatory system cells, muscles cells, connective tissue cells, and more). Pluripotent stem cells can be induced pluripotent stem cells (iPSCs) or embryonic stems cells (ESCs). Because they can propagate indefinitely and give rise to every cell type in the body, they represent a potential source for the development of therapeutic cells. Among many other, PSCs can be differentiated into hematopoietic progenitor cells, with the potential to generate any cell type from the hematopoietic cell lineage.

Hematopoietic stem cells (HSCs) are the stem cells that give rise to other blood cells through hematopoiesis. In adults, hematopoiesis occurs in the red bone marrow, in the core of most bones. The red bone marrow is derived from the mesoderm. During hematopoiesis, all mature blood cells are produced. It must balance production needs (the average person produces more than 500 billion blood cells every day) with the need to regulate the number of each blood cell type in the circulation. Hematopoietic stem cells give rise to different types of blood cells: myeloid and lymphoid cells. Myeloid and lymphoid lineages both are involved in dendritic cell formation. Myeloid cells include monocytes, macrophages, neutrophils, basophils, eosinophils, erythrocytes, and megakaryocytes to platelets. Lymphoid cells include T cells, B cells, natural killer cells, and innate lymphoid cells. The hematopoietic tissue contains cells with long-term and short-term regeneration capacities and committed multipotent, oligopotent, and unipotent progenitors. Hematopoietic stem cells constitute 1:10,000 of cells in myeloid tissue. In clinical settings, HSCs are used in HSC transplants in the treatment of cancers and other immune system disorders.

Natural killer cells, also known as NK cells or large granular lymphocytes (LGL), are a type of cytotoxic lymphocyte critical to the innate immune system that belong to the rapidly expanding family of known innate lymphoid cells (ILC) and represent 5-20% of all circulating lymphocytes in humans. The role of NK cells is analogous to that of cytotoxic T cells in the vertebrate adaptive immune response. NK cells provide rapid responses to virus-infected cell and other intracellular pathogens acting at around 3 days after infection and respond to tumor formation. Typically, immune cells detect the major histocompatibility complex (MHC) presented on infected cell surfaces, triggering cytokine release, causing the death of the infected cell by lysis or apoptosis. NK cells are unique, however, as they have the ability to recognize and kill stressed cells in the absence of antibodies and MHC, allowing for a much faster immune reaction. They were named “natural killers” because of the notion that they do not require activation to kill cells that are missing “self” markers of MHC class 1. This role is especially important because harmful cells that are missing MHC I markers cannot be detected and destroyed by other immune cells, such as T lymphocyte cells.

In addition to natural killer cells being effectors of innate immunity, both activating and inhibitory NK cell receptors play important functional roles, including self-tolerance and the sustaining of NK cell activity. NK cells also play a role in the adaptive immune response: numerous experiments have demonstrated their ability to readily adjust to the immediate environment and formulate antigen-specific immunological memory, fundamental for responding to secondary infections with the same antigen. The role of NK cells in both the innate and adaptive immune responses is becoming increasingly important in research using NK cell activity as a potential cancer therapy.

There is an unmet need in the art to develop methods for PSC differentiation that are efficient and yield pure cell cultures in a short period of time using convenient culture conditions.

### SUMMARY OF THE INVENTION

The present invention is based on the seminal discovery that a combination of an endothelial induction cocktail including a bone morphogenic protein (BMP) and a WNT signaling activator yields endothelial-like precursor cells displaying hybrid features of endothelial and hematopoietic precursor cells that can further yield terminally differentiated hematopoietic cells, such as NK cells. The combination of factors can be used to generate hematopoietic stem cells.

In one embodiment, the present invention provides a method of producing a population of CD34⁺ hematopoietic precursor cells including: a) contacting a culture of pluripotent stem cells (PSCs) with a WNT signaling pathway activator and a bone morphogenetic protein (BMP), wherein the PSCs are grown on a substrate for about 1-8 days; and b) contacting the culture of PSCs with a vascular endothelial growth factor (VEGF) alone or in combination with a WNT signaling pathway activator and/or a BMP for about 1-8 days following step a), wherein the cells produced after step b) are at least about 80% enriched for CD34⁺ cells in the total population of cells, thereby producing a population of CD34⁺ precursor cells.

In one aspect, contacting the adherent culture of PSCs with a WNT signaling pathway activator, a BMP, and/or a VEGF generates CD34⁺ hemogenic endothelium (HE). In another aspect, the method further includes contacting the population of CD34⁺ precursor cells with a mixture of agents to induce differentiation of the CD34⁺ precursor cells to terminally differentiated cells of the hematopoietic lineage. In some aspects, the cell of the hematopoietic lineage is a natural killer (NK) cell or other immune cell. In one aspect, the culture of PSCs is contacted with a WNT signaling pathway activator and a BMP for about 3 days and with a VEGF for about 4 additional days. In some aspects, the cells treated for about 1 week are CD34⁺, KDR⁺, CD31⁺ and CD45⁻. In other aspects, the CD34⁺ cells are also CD144⁺.

In another embodiment, the invention provides a method of producing natural killer (NK) cells including: a) contacting a culture of PSCs with a WNT signaling pathway activator and/or a BMP, wherein the PSCs are grown on a substrate for about 1-8 days; b) contacting the culture of PSCs with a VEGF alone or in combination with a WNT signaling pathway activator and/or a BMP for about 1-8 days following step a), thereby generating a population of CD34⁺ precursor cells, wherein the cells produced after step b) are at least about 80% enriched for CD34⁺ cells in the total population of cells; and c) contacting the population of CD34⁺ precursor cells with one or more of interleukin-7 (IL-7), IL-15, SCF and FMS-like tyrosine kinase 3 ligand (FLT3L), thereby producing NK cells.

In one aspect, contacting the population of CD34⁺ precursor cells of c) includes: (i) contacting the CD34⁺ precursor cells with IL-7, IL-15, SCF and FLT3L for about 5-10 days; and (ii) contacting the CD34⁺ precursor cells with IL-7, IL-15, FLT3L and SCF for at least about 7-21 days. In another aspect, the cells are transiently CD34⁺ and CD45⁺. In one aspect, contacting the adherent culture of PSCs includes one or more agents selected from about 1-10 μM WNT signaling pathway activator, about 5-50 ng/ml BMP, about 50-500 ng/ml VEGF. In some aspects, the WNT signaling pathway activator is a GSK3 inhibitor. In various aspects, the GSK3 inhibitor is CHIR99021. In other aspects, the BMP is BMP4. In some aspects, the VEGF is VEGF-A. In another aspect, contacting the adherent culture of PSCs includes about 8 μM CHIR99021, about 25 ng/ml BMP4 and/or about 200 ng/ml VEGFA. In one aspect, the CD34⁺ precursor cells are contacted with about 4-40 ng/ml IL-7, about 2-20 ng/ml IL-15, about 4-40 ng/mi SCF and/or about 1-20 ng/mi FLT3L. In various aspects, the CD34⁺ precursor cells are contacted with about 20 ng/mi IL-7, about 10 ng/ml IL-15, about 20 ng/mi SCF and/or about 10 ng/mi FLT3L. In one aspect, contacting the adherent culture of PSCs of a) with a WNT signaling pathway activator and a BMP is for about 2-5 days. In another aspect, the PSCs are subsequently contacted with a VEGF for about 2-5 days. In one aspect, contacting the population of CD34⁺ precursor cells with IL-7, IL-15, SCF and/or FLT3L is for about 5-10 days followed by contacting the population of CD34⁺ precursor cells with IL-7, IL-15, FLT3L and/or SCF is for at least about 7-21 days. In another aspects, the culture of PSCs is an adherent layer of cells. In some aspects, the layer of cells is grown in a two-dimensional culture system or on microcarriers. In other aspects, the PSCs are cultured on a coated surface including a laminin coating. In one aspect, the NK cells are further collected in suspension in a cell culture medium. In another aspect, the PSCs are human PSCs (hPSCs). In some aspects, the hPSCs are human induced pluripotent stem cells (hiPSCs) or human embryonic stems cells (hESCs). In various aspects, the CD34⁺ precursor cell is a CD34⁺ endothelial-like precursor cell. In one aspect, the NK cells are at least about 80% enriched.

In an additional embodiment, the invention provides a method of inducing NK cell differentiation from PSCs including: a) generating CD34⁺ hemogenic endothelium (HE) cells by: (i) contacting an adherent culture of PSCs with a WNT signaling pathway activator and a BMP for about 3 days; and (ii) contacting the adherent culture of PSCs of i) with a VEGF for about 4 days; thereby generating a population of cells including at least 80% CD34⁺ HE cells; b) contacting the CD34⁺ HE cells of a) with one or more of IL-7, IL-15, SCF and/or FLT3L for about 7 days, thereby generating a transient population of cells including at least 80% CD34⁺/CD45⁺ hematopoietic stem cells (HSCs); and c) subsequently contacting the CD34⁺, CD45⁺ HSCs of b) with one or more of IL-7, IL-15, FLT3L and SCF for at least about 7-21 days, thereby inducing NK cell differentiation from PSCs.

In one aspect, differentiated NK cells are CD56⁺, NKp30⁺, NKp44⁺, NKp46⁺, NKG2D⁺, NKG2A+, KIR2D⁺ and/or CD16⁺. In another aspect, the differentiated NK cells are CD56^{bright} or CD56^{dim}. In various aspects, the differentiated NK cells are cytotoxic NK cells.

In a further embodiment, the invention provides a kit including: a) an HE induction cocktail including: a WNT signaling pathway activator, a BMP and/or a VEGF, b) a NK induction cocktail including IL-7, IL-15, SCF and/or FLT3L; and c) instructions for inducing pluripotent stem cell (PSC) differentiation into NK cells.

In one aspect, the kit further includes a laminin-coated surface.

In one embodiment, the invention provides a method of generating terminally differentiated hematopoietic cells from PSCs including: a) generating CD34⁺ hematopoietic precursor cells by: (i) contacting an adherent culture of PSCs with a WNT signaling pathway activator and a BMP, wherein the PSCs are grown on a substrate for about 2-5 days; and (ii) contacting the adherent culture of PSCs with a VEGF for about 2-5 days following step (i), wherein the cells produced after step (ii) are at least about 80% enriched for CD34⁺ cells in the total population of cells; thereby generating CD34⁺ precursor cells; and b) contacting the CD34⁺ precursor cells of a) with a mixture of agents to induce differentiation of the CD34⁺ precursor cells into terminally differentiated hematopoietic cells, thereby generating terminally differentiated hematopoietic cells.

In one aspect, the terminally differentiated hematopoietic cell is an immune cell or a red blood cell. In various aspects, the immune cell is selected from the group consisting of macrophage, T cell, and NK cell.

In another embodiment, the invention provides a method of producing a population of CD34⁺ hematopoietic stem cells including a) contacting a culture of pluripotent stem cells (PSCs) with a WNT signaling pathway activator and a bone morphogenetic protein (BMP), wherein the PSCs are grown on a substrate for about 1-8 days; and b) contacting the culture of PSCs with a vascular endothelial growth factor (VEGF) alone or in combination with a WNT signaling pathway activator and/or a BMP for about 1-8 days following step a), wherein the cells produced after step b) are at least about 80% enriched for CD34+ hematopoietic stem cells in the total population of cells, thereby producing a population of CD34⁺ hematopoietic stem cells.

In one aspect, contacting the adherent culture of PSCs with a WNT signaling pathway activator, a BMP, and/or a VEGF generates CD34⁺ hemogenic endothelium (HE). In another aspect, the PSCs are human PSCs (hPSCs). In some aspects, the hPSCs are human induced pluripotent stem cells (hiPSCs) or human embryonic stems cells (hESCs).

In a further embodiment, the invention provides a method of generating hematopoietic stem cells from pluripotent stem cells (PSCs) including a) contacting an adherent culture of induced pluripotent stem cells (iPSCs) with a WNT signaling pathway activator and a bone morphogenetic protein (BMP), wherein the PSCs are grown on a substrate for about 2-5 days; and b) contacting the adherent culture of PSCs with a vascular endothelial growth factor (VEGF) for about 2-5 days following step (a), wherein the cells produced after step (b) are at least about 80% enriched for CD34⁺ cells in the total population of cells; thereby generating CD34⁺ precursor cells, thereby generating hematopoietic stem cells.

In one aspect, the iPSCs are human iPSCs (hiPSCs).

### BRIEF DESCRIPTION OF THE DRAWINGS

FIG. 1 is an illustrative schematic representation of a method of the invention.

FIGS. 2A-2C are illustrative schematic representations of the methods of the invention. FIG. 2A illustrates a method of the invention. FIG. 2B illustrates a method of the invention where the cells are cultured in a stem cell medium during the first 7 days of induction. FIG. 2C illustrates a method of the invention where the cells are cultured in the stem cell medium, a defined serum-free and animal-component-free PSC differentiation medium or an hPL-containing medium.

FIGS. 3A-3E illustrate cell characterization and survival in different methods of the inventions. FIG. 3A is a line graph illustrating the expression of an HSC marker (CD34), a leukocyte marker (CD45) and an endothelial marker (CD144) by the pluripotent stem cells during the course of the induction protocol. FIG. 3B is a line graph illustrating the expression of endothelial and hematopoietic markers in adherent cells and in cells in suspension during the course of the induction protocol. FIG. 3C is a line graph illustrating variation in the number of cells in suspension based on the stem cell medium, defined medium or hPL-containing medium used for cell differentiation. FIG. 3D is a line graph illustrating cell viability in different medium used for cell differentiation (stem cell basal medium, defined medium or hPL-containing medium). FIG. 3E is a line graph illustrating differences in the number of cells in suspension based on the day of cell transfer.

FIGS. 4A-4F are photographs illustrating bright light imaging of the cells during the course of the induction protocol. FIG. 4A shows the cells at day 0. FIG. 4B shows the cells at day 3. FIG. 4C shows the cells at day 7. FIG. 4D shows the cells at day 10. FIG. 4E shows the cells at day 14. FIG. 4F shows the cells at day 21.

FIG. 5 is a photograph illustrating colonies produced by the cells isolated on day 14, using a clonogenic assay kit.

FIGS. 6A-6C are bar graphs illustrating the percentage of colony forming cells (CFC) in control conditions (including all the induction factors), in the absence of IL15 (−IL15), and in only SCF. FIG. 6A is a bar graph illustrating the percentage of CFC at day 14. FIG. 6B is a bar graph illustrating the percentage of CFC at day 21. FIG. 6C is a bar graph illustrating the percentage of CFC at day 28.

FIG. 7 shows photographs illustrating the size of the colonies in the conditions described in FIG. 6, at day 21 (upper row) and at day 28 (lower row).

FIG. 8 is a photograph illustrating the cells after 14 days in culture in standard differentiation conditions. The round cells are cells budding off from the adherent cells to become HSCs, i.e., suspension cells transitioning into the supernatant, similar to hematopoiesis in vivo.

FIGS. 9A-9C is a graph illustrating characterization of the differentiated cells. FIG. 9A is a graph illustrating percent CD56⁺ cells. FIG. 9B is a graph illustrating CD16 and CD56 protein expression in the differentiated cells. FIG. 9C is a graph illustrating CD3 protein expression in differentiated cells.

FIGS. 10A-10B illustrate CD56 expression in cells over time. FIG. 10A is a line graph illustrating percent CD56 expression over time. FIG. 10B is a line graph illustrating differences in CD56 expression by the cells based on the medium used for cell differentiation (stem cell medium, defined medium or hPL-containing medium).

FIG. 11 is a graph illustrating the total number of cells in suspension, including NK cells over time.

FIGS. 12A-12B are graphs bar illustrating percent caspase 3/7⁺ or dead K562 cells after incubation with the NK cells for 4 hours. FIG. 12A is a graph bar illustrating percent of cells after incubation with NK cells obtained after 35 days of differentiation. FIG. 12A is a graph bar illustrating percent of caspase 3/7⁺ or dead K562 cells after 4 hours incubation with NK cells obtained after 56 days of differentiation.

FIGS. 13A-13I are graphs illustrating protein expression in the differentiated NK cells as measured by flow cytometry. FIG. 13A is a graph confirming that the differentiated cells are NK cells, as shown by the CD56 expression. FIG. 13B is a graph illustrating CD56 and CD16 protein expressions. FIG. 13C is a graph illustrating NKp30 protein expression. FIG. 13D is a graph illustrating NKp44 protein expression. FIG. 13E is a graph illustrating NKp46 protein expression. FIG. 13F is a graph illustrating NKG2D protein expression. FIG. 13G is a graph illustrating NKG2A protein expression. FIG. 13H is a graph illustrating NKG2C protein expression. FIG. 13I is a graph illustrating KIR3D and KIR2D protein expressions.

FIGS. 14A-14J are graphs illustrating differences of protein expression in CD56^{bright} and CD56^{dim} NK cells. FIG. 14A is a graph illustrating percent of NK cells expressing CD56^{bright} (top) and CD56^{dim} (bottom). FIG. 14B is a graph illustrating CD56 and CD16 expressions in CD56^{bright} (top) and CD56^{dim} (bottom) NK cells from cells gated as indicated in FIG. 14A (arrow). FIG. 14C is a graph illustrating side and forward scatter analysis in CD56^{bright} (top) and CD56^{dim} (bottom) NK cells. FIG. 14D is a graph illustrating CD56 expression in CD56^{bright} (top) and CD56^{dim} (bottom) NK cells from the isolated population illustrated in FIG. 14C (right arrow). FIG. 14E is a graph illustrating NKp46 expression in CD56^{bright} (top) and CD56^{dim} (bottom) NK cells. FIG. 14F is a graph illustrating NKp30 in CD56^{bright} (top) and CD56^{dim} (bottom) NK cells. FIG. 14G is a graph illustrating percNKp44 in CD56^{bright} (top) and CD56^{dim} (bottom) NK cells. FIG. 14H is a graph illustrating NKG2D in CD56^{bright} (top) and CD56^{dim} (bottom) NK cells. FIG. 14I is a graph illustrating NKG2A in CD56^{bright} (top) and CD56^{dim} (bottom) NK cells. FIG. 14J is a graph illustrating NKG2C in CD56^{bright} (top) and CD56^{dim} (bottom) NK cells.

FIGS. 15A-15B illustrate the use of a method of the invention to generate r cells. FIG. 15A is an illustrative schematic representation of a method of the invention to generate T cells. FIG. 15B shows graphs illustrating the flow cytometry profile of T cells.

FIGS. 16A-16B illustrate the use of a method of the invention to generate macrophages. FIG. 16A is an illustrative schematic representation of a method of the invention to generate macrophages. FIG. 16B shows graphs illustrating the flow cytometry profile of macrophages.

FIG. 17 is an illustrative schematic representation of a method of the invention to generate hemogenic endothelium (HE).

FIGS. 18A-18B illustrate the number of cells harvested and number of colonies obtained from HE after 14 and 17 days. FIG. 18A is a graph illustrating the number of HSC cells obtained from HE after 14 and 17 days. FIG. 18B is a graph illustrating the number of HSC colonies obtained from HE after 14 and 17 days.

FIGS. 19A-19B illustrate the cytometry analysis of the HSC cells harvested at day 14 and 17. FIG. 19A illustrates the CD34, CD90, CD38 and CD45 cytometry analysis of the HSC cells harvested at day 14. FIG. 19B illustrates the CD34, CD90, CD38 and CD45 cytometry analysis of the HSC cells harvested at day 17.

FIG. 20 is an illustrative schematic representation of a method of the invention to generate hemogenic endothelium (HE) and HSC therefrom in various cell culture media.

FIG. 21 is a graph illustrating NK cell count after HSC cryopreservation.

### DETAILED DESCRIPTION OF THE INVENTION

The present invention is based on the seminal discovery that a combination of an endothelial induction cocktail bone morphogenic protein (BMP) and WNT signaling activator yields endothelial-like precursor cells displaying hybrid features of endothelial and hematopoietic precursor cells that further yield terminally differentiated hematopoietic cells such as NK cells. The combination of factors can be used to generate hematopoietic stem cells.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a,” “an,” and “the” include plural references unless the context clearly dictates otherwise. Thus, for example, references to “the method” includes one or more methods, and/or steps of the type described herein which will become apparent to those persons skilled in the art upon reading this disclosure and so forth.

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, it will be understood that modifications and variations are encompassed within the spirit and scope of the instant disclosure. The preferred methods and materials are now described.

In one embodiment, the present invention provides a method of producing a population of CD34⁺ hematopoietic precursor cells including: a) contacting a culture of pluripotent stem cells (PSCs) with a WNT signaling pathway activator and a bone morphogenetic protein (BMP), wherein the PSCs are grown on a substrate for about 1-8 days; and b) contacting the culture of PSCs with a vascular endothelial growth factor (VEGF) alone or in combination with a WNT signaling pathway activator and/or a BMP for about 1-8 days following step a), wherein the cells produced after step b) are at least about 80% enriched for CD34⁺ cells in the total population of cells, thereby producing a population of CD34⁺ precursor cells.

The methods described herein describe cellular culture conditions in which human pluripotent stem cells are grown, that yield the generation of a population of CD34⁺ hematopoietic precursor cells.

Stem cells are undifferentiated cells that have the ability to self-renew indefinitely and to remain in said undifferentiated state. As opposed to embryonic stem cells which can only be isolated from the inner mass of a blastocyst, there are three known accessible sources of adult stem cells: the bone marrow which requires the drilling of a bone, the adipose tissue which is accessible by liposuction, and the blood, from which the cells can be extracted among other cells. The term “pluripotent stem cells,” as used herein refers to cells that are capable of generating all the cell types of an organism, i.e., cells derived from any of the three germ layers. On the other hand, multipotent stem cells can differentiate into several cell type, but only those of a closely related family of cells, generally the cell types of the organ from which they originate. Most adult stem cells are multipotent but small amounts of pluripotent adult stem cells can be retrieved from umbilical cord or other tissues. The sources of cells used for cell therapy include stem cells such as embryonic stem cells (ESCs), adult stem cells, and induced pluripotent stem cells (iPSCs).

In some aspects, the PSCs used in the methods described herein are human (hPSCs), and in some instances the human PSCs are induced pluripotent stem cells (hiPSCs) or human embryonic stems cells (hESCs).

By “generating” or “producing” CD34⁺ hematopoietic precursor cells, it is meant that the present methods provide physical and chemical culture conditions that have been optimized to induce the differentiation of PSCs into CD34⁺ hematopoietic precursor cells. The differentiation method described herein yields a CD34⁺ hematopoietic precursor cell population that is enriched for CD34⁺ hematopoietic precursor cells. For example, greater than 80%, greater than 85%, greater than 90%, greater than 95%, 96%, 97%, 98% or 99% CD34⁺ hematopoietic precursor cells are obtained in short times and using convenient cultures conditions.

Physical culture conditions include but are not limited to the culture environment of the cell (e.g., adherent versus suspension culture, or in two-dimensional versus in three-dimensional culture systems), the pH of the culture medium, the gas concentration in the incubator (e.g., CO₂ concentration, O₂ concentration), and the temperature.

There are two basic systems for growing cells in culture, as monolayers on an artificial substrate (i.e., adherent culture) or free-floating in the culture medium (suspension culture). The majority of the cells derived from vertebrates, with the exception of hematopoietic cell lines and a few others, are anchorage-dependent and have to be cultured on a suitable substrate that is specifically treated to allow cell adhesion and spreading (i.e., tissue-culture treated). However, many cell lines can also be adapted for suspension culture.

In another aspect, the culture of PSCs is an adherent layer of cells. In some aspects, the layer of cells is grown in a two-dimensional culture system or on microcarriers.

In addition to the treatment of the tissue-culture surface, cells can require to be grown on coated surfaces, to enhance or improve their adhesion and/or spreading (i.e., using a coating). “Coating” as an additional surface treatment stands for all additional modifications made to increase cell adhesion in addition to the standard plasma or corona treatment which is performed on all cell culture plastic by manufacturer. Usually, coating is done with proteins or peptides. Various proteins can be used to coat tissue-culture treated dishes, including poly-L-Lysine, poly-D-Lysine, poly-Omithine, gelatin, collagen I, IV, fibronectin, laminin, vitronectin, osteopontin, fibronectin domains, Matrigel™ (several components of the extracellular matrix with bound growth factors etc.), collagen gels, alginate gels, and lactate gels.

In other aspects, the PSCs are cultured on a coated surface including a laminin coating.

Physical culture conditions include the gas concentration in the incubator. Incubation of cell cultures is typically performed in normal atmosphere with 15-22% oxygen and 5% CO₂ for expansion and seeding. In various aspects, the PSCs are grown in a humidified atmosphere including about 5% CO₂ concentration, and normoxic conditions (non-hypoxic O₂ concentration). While hypoxic culture conditions are thought to support stem cell performance in general, in the present methods, the PSCs are cultured under conditions that are not hypoxic conditions. As used herein, “normoxic” conditions refer to culture conditions including atmospheric O₂ concentration (e.g., about 15-25% O₂ concentration). As used herein, hypoxic conditions are characterized by a lower oxygen concentration as compared to the oxygen concentration of ambient air (approximately 15%-25% oxygen).

Chemical culture conditions include but are not limited to the agents or molecules that are added to the culture medium to achieve the desired effects sought after (i.e., differentiation of PSCs into CD34⁺ hematopoietic precursor cells). The terms “agent” and “molecule” are used interchangeably and include, but are not limited to, small molecules (including small molecules that do not have optimal cell-permeability), lipids, nucleosides, nucleotides, nucleic acids, polynucleotides, oligonucleotides, antibodies, toxins, negatively charged polymers and other polymers, for example proteins, peptides, hormones, carbohydrates, or polyamines.

In the methods described herein, pluripotent stem cells are differentiated into CD34⁺ hematopoietic precursor. As used herein, “CD34⁺ hematopoietic precursor” or “CD34⁺ hematopoietic progenitor” refers to transient cells that express some of hematopoietic stem cells (HSCs) markers, and display some of their characteristics, without being presenting all the HSCs markers and characteristics. For example, HSCs are usually characterized as CD34⁺/CD45⁺ non-adherent cells. After about 7 days of culture under the conditions described herein, the CD34⁺ hematopoietic precursor cells are CD34⁺ but they remain CD23⁺ and CD45⁻, they are also still adherent cells.

Unless otherwise specifically describes, pluripotent stem cells are maintained in a stem cell medium, suitable for the culture and propagation of pluripotent stem cells. Stem cell basal are well known in the art; non-limiting examples of such suitable media include but are not limited to StemPro34™. For HSC differentiation, the pluripotent stem cells are switched to a “defined medium”. As used herein, the term “basal medium” generally refers to a base medium, without any additives added by the used (for example, a basal medium refers to a medium as commercially available). Those generally include water, nutrients, salts and amino acids, but no additive or supplements. A basal medium can be supplemented with general additives to obtain a “supplemented basal media”. Non-limiting examples of supplements include but are not limited to insulin or ascorbate for example. A basal medium can also be completed with specific signaling molecules such as those identified by a user as necessary to achieve a particular goal with the cell culture, such as for example driving the differentiation of a cell type of interest into a target cell type. Such complete basal medium can be referred to as a “final”, “complete” or “cell-specific” medium.

In the context of the present invention, the cell culture media are additionally referred to based on their use (e.g., to generate HE, or to induce the differentiation of cells). For example, the terms “defined basal medium” and “defined medium” are meant to refer to ready to use medium formulations for the generation of HE, that only contains specific components at quantifiable amounts, e.g., they do not contain blood serum or components directly isolated from it or other animal or tissue-derived products isolated from organisms or cells. Such ready to use formulations can be found in commercially available products, or in “in-house” compositions developed by users. Without wishing to be limited to any specific formulation, it is provided that a general formulation for a defined medium can include: a basal medium such as DMEM, DMEM/F12, IMDM, or mixture thereof and supplements comprising one of more of the following without being limited to: insulin (optionally combined with transferrin and selenium), serum albumin (preferably human recombinant), polyvinylalcohol (PVA), lipids/fatty acids, glutamine/alanyl-glutamine (Glutamax)/amino acids in general, antioxidants such as ascorbic acid/ascorbic acid-2-phosphate or thiol compounds, and inorganic salts (for the supplemented version). Non-limiting examples of commercially available defined basal media include but are not limited to:
APEL™ including 1× Iscove's modifid Dulbecco's medium (IMDM), 1× Ham's F-12 nutrient mixture, Albucult (rh Albumin)(5 mg/ml), Polyvinylalcohol (PVA), Linoleic acid (100 ng/ml), Linolenic acid (100 ng/ml), SyntheChol (synthetic cholesterol) (2.2 mg/ml), a-Monothioglycerol (a-MTG) (3.9 ml per 100 ml), rh Insulin-transferrin-selenium-ethanolamine solution (rhITS-Eth), protein-free hybridoma mixture II (PFHMII) (5%), ascorbic acid 2 phosphate (50 μg/ml), GlutamaxI (L-alanyl-L-glutamine) (2 mM) and penicillin/streptomycin (50 U Pen G/50 mg streptomycin sulfate);
APELII™ or APEL2™ including 1× Iscove's modifid Dulbecco's medium (IMDM), 1× Ham's F-12 nutrient mixture, Albucult (rh Albumin) (5 mg/ml), Polyvinylalcohol (PVA), Linoleic acid (100 ng/ml), Linolenic acid (100 ng/ml), SyntheChol (synthetic cholesterol) (2.2 mg/ml), a-Monothioglycerol (a-MTG) (3.9 ml per 100 ml), rh Insulin-transferrin-selenium-ethanolamine solution (rhITS-Eth), ascorbic acid 2 phosphate (50 μg/ml), GlutamaxI(L-alanyl-L-glutamine)(2 mM) and penicillin/streptomycin (50 U Pen G/50 mg streptomycin sulfate);
APALII including 1× Iscove's modifid Dulbecco's medium (IMDM), Polyvinylalcohol (PVA, 0.1%), Albucult (rh Albumin) (0.2%), ascorbic acid 2 phosphate (250 uM), lipids (1%), rh Insulin-transferrin-selenium-ethanolamine solution (rhITS-Eth, 0.1%).
ESSENTIAL 6™ or E6™ including DMEM/F12, insulin, transferrin, sodium selenium, ascorbic acid-2-phosphate, and NaHCO3; and
ESSENTIAL 8™ or E8™ including DMEM/F12, L-ascorbic acid-2-phosphate magnesium (64 mg/4), sodium selenium (14 μg/l), insulin (19.4 mg/l), NaHCO3 (543 mg/1) and transferrin (10.7 mg/I).

Such basal or supplemented defined media, used for HE generation can then be completed with small molecules of interest as needed.

By “contacting” it is meant that the cells are cultured with one or more agent of interest, added in the defined basal or supplemented medium (or as detailed below in the hPL-containing basal or supplemented medium). That is the cells are cultured in their regular culture basal or supplemented medium, in which a desired concentration of one or more agent of interest is added. For examples, the cells are cultured with a WNT signaling pathway activator, a BMP, and/or a VEGF.

A “pathway signaling activator” as used herein refers to any molecule that is capable of activating, enhancing, or inducing a signaling pathway of interest. A signaling pathway is a series of chemical reactions in which a group of molecules in a cell work together to control a cell function, such as cell differentiation. A cell receives signals from its environment when a molecule, such as a hormone or growth factor, binds to a specific protein receptor on or in the cell. After the first molecule in the pathway receives a signal, it activates another molecule. This process is repeated through the entire signaling pathway until the last molecule is activated and the cell function is carried out. Abnormal activation of signaling pathways, or inhibition of a signaling pathway may lead to diseases, or, in the case of pluripotent cells to alteration of the pluripotent state, and therefore to differentiation. The term “molecule” includes, but is not limited to, small molecules (including small molecules that do not have optimal cell-permeability), lipids, nucleosides, nucleotides, nucleic acids, polynucleotides, oligonucleotides, antibodies, toxins, negatively charged polymers and other polymers, for example proteins, peptides, hormones, carbohydrates, or polyamines. Non-limiting examples of polynucleotides include short interfering nucleic acid (siNA), antisense, enzymatic nucleic acid molecules, 2′,5′-oligoadenylate, triplex forming oligonucleotides, aptamers, and decoys. Biologically active molecules include antibodies (e.g., monoclonal, chimeric, humanized etc.), cholesterol, hormones, antivirals, peptides, proteins, chemotherapeutics, small molecules, vitamins, co-factors, nucleosides, nucleotides, oligonucleotides, enzymatic nucleic acids, antisense nucleic acids, triplex forming oligonucleotides, 2,5-A chimeras, allozymes, aptamers, decoys and analogs thereof, and small nucleic acid molecules, such as short interfering nucleic acid (siNA), short interfering RNA (siRNA), double-stranded RNA (dsRNA), micro-RNA (miRNA), antagomirs, and short hairpin RNA (shRNA) molecules.

The WNT signaling pathways are a group of signal transduction pathways which begin with proteins that pass signals into a cell through cell surface receptors. Wnt signaling pathways use either nearby cell-cell communication (paracrine) or same-cell communication (autocrine). Three Wnt signaling pathways have been characterized: the canonical Wnt pathway, the noncanonical planar cell polarity pathway, and the noncanonical Wnt/calcium pathway. All three pathways are activated by the binding of a Wnt-protein ligand to a Frizzled family receptor, which passes the biological signal to the Dishevelled protein inside the cell. The canonical Wnt pathway leads to regulation of gene transcription and is thought to be negatively regulated in part by the SPATS1 gene. The noncanonical planar cell polarity pathway regulates the cytoskeleton that is responsible for the shape of the cell. The noncanonical Wnt/calcium pathway regulates calcium inside the cell. Wnt signaling was first identified for its role in carcinogenesis, then for its function in embryonic development. The embryonic processes it controls include body axis patterning, cell fate specification, cell proliferation and cell migration. These processes are necessary for proper formation of important tissues including bone, heart, and muscle. Its role in embryonic development was discovered when genetic mutations in Wnt pathway proteins produced abnormal fruit fly embryos. Later research found that the genes responsible for these abnormalities also influenced breast cancer development in mice. Wnt signaling also controls tissue regeneration in adult bone marrow, skin, and intestine.

In some aspects, the WNT signaling pathway activator a GSK3 inhibitor. In various aspects, the GSK3 inhibitor is CHIR99021.

The transforming growth factor beta (TGF-beta) superfamily includes TGF-beta proteins, bone morphogenetic proteins (BMPs), growth differentiation factors (GDFs), glial-derived neurotrophic factors (GDNFs), Activins, Inhibins, Nodal, Lefty, and Müllerian inhibiting substance (MIS). Bone morphogenetic proteins (BMPs) are a group of growth factors also known as cytokines and as metabologens. Originally discovered by their ability to induce the formation of bone and cartilage, BMPs are now considered to constitute a group of pivotal morphogenetic signals, orchestrating tissue architecture throughout the body. The important functioning of BMP signals in physiology is emphasized by the multitude of roles for dysregulated BMP signaling in pathological processes.

BMPs interact with specific receptors on the cell surface, referred to as bone morphogenetic protein receptors (BMPRs). Signal transduction through BMPRs results in mobilization of members of the SMAD family of proteins. The signaling pathways involving BMPs, BMPRs and SMADs are important in the development of the heart, central nervous system, and cartilage, as well as post-natal bone development. They have an important role during embryonic development on the embryonic patterning and early skeletal formation. As such, disruption of BMP signaling can affect the body plan of the developing embryo. For example, BMP4 and its inhibitors noggin and chordin help regulate polarity of the embryo (i.e., back to front patterning). Specifically, BMP-4 and its inhibitors play a major role in neurulation and the development of the neural plate. BMP-4 signals ectoderm cells to develop into skin cells, but the secretion of inhibitors by the underlying mesoderm blocks the action of BMP-4 to allow the ectoderm to continue on its normal course of neural cell development.

In one aspect, the BMP is BMP4.

Vascular endothelial growth factor (VEGF), originally known as vascular permeability factor (VPF), is a signal protein produced by many cells that stimulates the formation of blood vessels. To be specific, VEGF is a sub-family of growth factors, the platelet-derived growth factor family of cystine-knot growth factors. They are important signaling proteins involved in both vasculogenesis (the de novo formation of the embryonic circulatory system) and angiogenesis (the growth of blood vessels from pre-existing vasculature). It is part of the system that restores the oxygen supply to tissues when blood circulation is inadequate such as in hypoxic conditions. Serum concentration of VEGF is high in bronchial asthma and diabetes mellitus. VEGF's normal function is to create new blood vessels during embryonic development, new blood vessels after injury, muscle following exercise, and new vessels (collateral circulation) to bypass blocked vessels.

In some aspects, the VEGF is VEGF-A.

In various aspects, the chemical culture conditions of the presently described methods include a mixture of agents including WNT signaling pathway activator, a BMP, and/or a VEGF.

For example, the mixture of agents includes a WNT signaling pathway activator and a BMP. In an additional example, the mixture of agents includes a WNT signaling pathway activator, a BMP, and a VEGF alone. In another example, the mixture of agents includes WNT signaling pathway activator alone, a BMP alone or a VEGF alone.

In one aspect, the PSCs are grown on a substrate in the presence of a WNT signaling pathway activator and a bone morphogenetic protein (BMP) for about 1-8 days. For example, the cells are grown for about 1, 2, 3, 4, 5, 6, 7, 8 or more days in the presence of a WNT signaling pathway activator and a BMP.

In another aspect, following the initial culture in the presence of a WNT signaling pathway activator and a BMP the PSCs are grown on a substrate in the presence of a VEGF for about 1-8 days. For example, following the initial culture the cells are grown for about 1, 2, 3, 4, 5, 6, 7, 8 or more days in the presence of a VEGF.

In yet another aspect, following the initial culture in the presence of a WNT signaling pathway activator and a BMP the PSCs are grown on a substrate in the presence of a VEGF, a WNT signaling activator and a BMP for about 1-8 days. For example, following the initial culture the cells are grown for about 1, 2, 3, 4, 5, 6, 7, 8 or more days in the presence of a VEGF, a WNT signaling activator and a BMP.

In one aspect, the culture of PSCs is contacted with a WNT signaling pathway activator and a BMP for about 3 days and then with a VEGF for about 4 additional days.

In one aspect, contacting the adherent culture of PSCs with a WNT signaling pathway activator, a BMP, and/or a VEGF generates CD34⁺ hemogenic endothelium (HE).

Hemogenic endothelium (HE) is constituted of a special subset of endothelial cells scattered within blood vessels that can differentiate into hematopoietic cells. The development of hematopoietic cells in the embryo proceeds sequentially from mesoderm through the hemangioblast to the hemogenic endothelium and hematopoietic progenitors. Hemangioblasts are the multipotent precursor cells that can differentiate into both hematopoietic and endothelial cells. Hemangioblasts are the progenitors that form the blood islands. Hemangioblasts have been first extracted from embryonic cultures and manipulated by cytokines to differentiate along either hematopoietic or endothelial route.

In exemplary aspects, contacting the population of CD34⁺ precursor cells further comprises contacting the cells with a transforming growth factor β (TGFβ)/SMAD2/SMAD3 pathway signaling inhibitor.

A “pathway signaling inhibitor” as used herein refers to any molecule that is capable of inhibiting a signaling pathway of interest. A signaling pathway is a series of chemical reactions in which a group of molecules in a cell work together to control a cell function, such as cell differentiation. A cell receives signals from its environment when a molecule, such as a hormone or growth factor, binds to a specific protein receptor on or in the cell. After the first molecule in the pathway receives a signal, it activates another molecule. This process is repeated through the entire signaling pathway until the last molecule is activated and the cell function is carried out. Abnormal activation of signaling pathways, or inhibition of a signaling pathway may lead to diseases, or, in the case of pluripotent cells to alteration of the pluripotent state, and therefore to differentiation.

A “TGFβ/SMAD2/SMAD3 pathway signaling inhibitor”, as used herein refer to any molecule capable of inhibiting the TGFβ/SMAD2/SMAD3. Signaling pathway inhibition is the opposite of signaling pathway upregulation. In this process, small molecules called “signal transduction inhibitors” or “pathway signaling inhibitors” block the communication between different molecules of the pathway and interrupt the molecular signaling cascade. TGFβ/SMAD2/SMAD3 pathway signaling inhibitors include for example, any molecule that inhibits TGFβ type 1 receptor (or ALK5), and its relatives ALK4 and ALK7. Non-limiting examples of TGFβ/SMAD2/SMAD3 pathway signaling inhibitor include SB431542, LY3200882, TP0427736 HCl, RepSox, SB525334, GW788388, BIBF-0775, SD-208, galunisertib, vactosertib, A-83-01, LY2109761, SB505124, LY364947 and LDN-212854.

In some aspect contacting the cells with a TGFβ/SMAD2/SMAD3 pathway signaling inhibitor comprises contacting the cells with about 1-25 μM TGFβ/SMAD2/SMAD3 pathway signaling inhibitor.

In one aspect, the TGFβ/SMAD2/SMAD3 pathway signaling inhibitor is SB431542.

In some aspects, the TGFβ/SMAD2/SMAD3 pathway signaling inhibitor is added to the culture at a concentration that ranges from about 5 μM to 20 μM. For example, the cells are grown in a culture medium that includes about 5, 6, 7, 8, 9, 10, 11, 12, 13, 14, 15, 16, 17, 18, 19, 20 μM or more. In one aspect, the mixture includes about 10 μM TGFβ/SMAD2/SMAD3 pathway signaling inhibitor. In another aspect, the mixture includes about 5-20 μM SB431542. In some aspects, the mixture includes about 8-15 μM SB431542. In other aspects, the mixture includes about 10 μM SB431542.

In another aspect, the method further includes contacting the population of CD34⁺ precursor cells with a mixture of agents to induce differentiation of the CD34⁺ precursor cells to terminally differentiated cells of the hematopoietic lineage.

“Terminally differentiated cells of the hematopoietic lineage” refers to any of the terminally differentiated cells that can emerge from one of the three blood cell lineages, which include erythroid, lymphoid, and myeloid lineages. Erythroid cells are the oxygen carrying red blood cells. Lymphoid cells are the cornerstone of the adaptive immune system, they are derived from common lymphoid progenitors. The lymphoid lineage is primarily composed of T-cells and B-cells and natural killer cells (i.e., white blood cells). Myeloid cells, which includes granulocytes, megakaryocytes, and macrophages, are derived from common myeloid progenitors, and are involved in such diverse roles as innate immunity, adaptive immunity, and blood clotting.

In some aspects, the cell of the hematopoietic lineage is a natural killer (NK) cell or other immune cell.

In some aspects, the cells treated for about 1 week are CD34⁺, KDR⁺, CD31⁺ and CD45⁻. In other aspects, the CD34⁺ cells are also CD144⁺.

In another embodiment, the invention provides a method of producing natural killer (NK) cells including: a) contacting a culture of PSCs with a WNT signaling pathway activator and/or a BMP, wherein the PSCs are grown on a substrate for about 1-8 days; b) contacting the culture of PSCs with a VEGF alone or in combination with a WNT signaling pathway activator and/or a BMP for about 1-8 days following step a), thereby generating a population of CD34⁺ precursor cells, wherein the cells produced after step b) are at least about 80% enriched for CD34⁺ cells in the total population of cells; and c) contacting the population of CD34⁺ precursor cells with one or more of interleukin-3 (IL-3), IL-7, IL-15, SCF and FMS-like tyrosine kinase 3 ligand (FLT3L), thereby producing NK cells.

In one embodiment, the invention provides a method of producing natural killer (NK) cells including: a) contacting a culture of PSCs with a WNT signaling pathway activator and/or a BMP, wherein the PSCs are grown on a substrate for about 1-8 days; b) contacting the culture of PSCs with a VEGF alone or in combination with a WNT signaling pathway activator and/or a BMP for about 1-8 days following step a), thereby generating a population of CD34⁺ precursor cells, wherein the cells produced after step b) are at least about 80% enriched for CD34⁺ cells in the total population of cells; and c) contacting the population of CD34⁺ precursor cells with one or more of IL-7, IL-15, SCF and FMS-like tyrosine kinase 3 ligand (FLT3L), thereby producing NK cells.

Natural killer cells, also known as NK cells or large granular lymphocytes (LGL), are a type of cytotoxic lymphocyte critical to the innate immune system that belong to the rapidly expanding family of known innate lymphoid cells (ILC) and represent 5-20% of all circulating lymphocytes in humans. They have different functions including: cytolytic granule mediated cell apoptosis, antibody-dependent cell-mediated cytotoxicity (ADCC) and cytokine-induced NK and cytotoxic T lymphocyte (CTL) activation.

NK cells are cytotoxic; small granules in their cytoplasm contain proteins such as perforin and proteases known as granzymes. Upon release in close proximity to a cell slated for killing, perforin forms pores in the cell membrane of the target cell, creating an aqueous channel through which the granzymes and associated molecules can enter, inducing either apoptosis or osmotic cell lysis. The distinction between apoptosis and cell lysis is important in immunology: lysing a virus-infected cell could potentially release the virions, whereas apoptosis leads to destruction of the virus inside. α-defensins, antimicrobial molecules, are also secreted by NK cells, and directly kill bacteria by disrupting their cell walls in a manner analogous to that of neutrophils.

Infected cells are routinely opsonized with antibodies for detection by immune cells. Antibodies that bind to antigens can be recognized by FcγRIII (CD16) receptors expressed on NK cells, resulting in NK activation, release of cytolytic granules and consequent cell apoptosis. This is a major killing mechanism of some monoclonal antibodies like rituximab (Rituxan), ofatumumab (Azzera), and others.

Cytokines play a crucial role in NK cell activation. As these are stress molecules released by cells upon viral infection, they serve to signal to the NK cell the presence of viral pathogens in the affected area. Cytokines involved in NK activation include IL-12, IL-15, IL-18, IL-2, and CCL5. NK cells are activated in response to interferons or macrophage-derived cytokines. They serve to contain viral infections while the adaptive immune response generates antigen-specific cytotoxic T cells that can clear the infection. NK cells work to control viral infections by secreting IFNγ and TNFα. IFNγ activates macrophages for phagocytosis and lysis, and TNFα acts to promote direct NK tumor cell killing. Patients deficient in NK cells prove to be highly susceptible to early phases of herpes virus infection.

Tumor-infiltrating NK cells have been reported to play a critical role in promoting drug-induced cell death in human triple-negative breast cancer. Since NK cells recognize target cells when they express non-self HLA antigens (but not self), autologous (patients' own) NK cell infusions have not shown any antitumor effects. Instead, investigators are working on using allogeneic cells from peripheral blood, which requires that all T cells be removed before infusion into the patients to remove the risk of graft versus host disease, which can be fatal. This can be achieved using an immunomagnetic column (CliniMACS). In addition, because of the limited number of NK cells in blood (only 10% of lymphocytes are NK cells), their number needs to be expanded in culture. This can take a few weeks and the yield is donor dependent.

Interleukin 3 (IL-3) is a protein that in humans is encoded by the IL3 gene, which is also referred to as colony-stimulating factor, multi-CSF, mast cell growth factor, MULTI-CSF, MCGF; MGC79398, or MGC79399. IL-3 is produced as a monomer by activated T cells, monocytes/macrophages, and stroma cells. The major function of IL-3 cytokine is to regulate the concentrations of various blood-cell types. It induces proliferation and differentiation in both early pluripotent stem cells and committed progenitors. It also has many more specific effects like the regeneration of platelets and potentially aids in early antibody isotype switching. IL-3 is capable of stimulating differentiation of immature myelomonocytic cells causing changes to the macrophage and granulocyte populations. IL-3 signaling is able to give rise to widest array of cell linages which is why it has been independently named “multi-CSF”. Interleukin 3 stimulates the differentiation of multipotent hematopoietic stem cells into myeloid progenitor cells or, with the addition of IL-7, into lymphoid progenitor cells. In addition, IL-3 stimulates proliferation of all cells in the myeloid lineage (granulocytes, monocytes, and dendritic cells), in conjunction with other cytokines, e.g., Erythropoietin (EPO), Granulocyte macrophage colony-stimulating factor (GM-CSF), and IL-6.

IL-7 is a hematopoietic growth factor secreted by stromal cells in the bone marrow and thymus. It is also produced by keratinocytes, dendritic cells, hepatocytes, neurons, and epithelial cells, but is not produced by normal lymphocytes. IL-7 stimulates the differentiation of multipotent (pluripotent) hematopoietic stem cells into lymphoid progenitor cells (as opposed to myeloid progenitor cells where differentiation is stimulated by IL-3 It also stimulates proliferation of all cells in the lymphoid lineage (B cells, T cells and NK cells). It is important for proliferation during certain stages of B-cell maturation, T and NK cell survival, development, and homeostasis.

Interleukin-15 (IL-15) is a cytokine with structural similarity to Interleukin-2 (IL-2). Like IL-2, IL-15 binds to and signals through a complex composed of IL-2/IL-15 receptor beta chain (CD122) and the common gamma chain (gamma-C, CD132). IL-15 is secreted by mononuclear phagocytes (and some other cells) following infection by virus(es). This cytokine induces the proliferation of natural killer cells, i.e., cells of the innate immune system whose principal role is to kill virally infected cells. IL-15 regulates the activation and proliferation of T and natural killer (NK) cells. Survival signals that maintain memory T cells in the absence of antigen are provided by IL-15. This cytokine is also implicated in NK cell development. In rodent lymphocytes, IL-15 prevents apoptosis by inducing BCL2L1/BCL-x(L), an inhibitor of the apoptosis pathway. In humans with celiac disease IL-15 similarly suppresses apoptosis in T-lymphocytes by inducing Bcl-2 and/or Bcl-xL.

Stem cell factor (also known as SCF, KIT-ligand, KL, or steel factor) is a cytokine that binds to the c-KIT receptor (CD117). SCF exist both as a transmembrane protein and a soluble protein and plays an important role in hematopoiesis, spermatogenesis, and melanogenesis.

SCF plays an important role in the hematopoiesis during embryonic development. Sites where hematopoiesis takes place, such as the fetal liver and bone marrow, all express SCF. SCF may serve as guidance cues that direct hematopoietic stem cells (HSCs) to their stem cell niche (the microenvironment in which a stem cell resides), and it plays an important role in HSC maintenance. SCF plays a role in the regulation of HSCs in the stem cell niche in the bone marrow. SCF has been shown to increase the survival of HSCs in vitro and contributes to the self-renewal and maintenance of HSCs in vivo. HSCs at all stages of development express the same levels of the receptor for SCF (c-KIT). The stromal cells that surround HSCs are a component of the stem cell niche, and they release a number of ligands, including SCF. In the bone marrow, HSCs and hematopoietic progenitor cells are adjacent to stromal cells, such as fibroblasts and osteoblasts. These HSCs remain in the niche by adhering to ECM proteins and to the stromal cells themselves. SCF has been shown to increase adhesion and thus may play a large role in ensuring that HSCs remain in the niche. SCF may be used along with other cytokines to culture HSCs and hematopoietic progenitors. The expansion of these cells ex vivo would allow advances in bone marrow transplantation, in which HSCs are transferred to a patient to re-establish blood formation. One of the problems of injecting SCF for therapeutic purposes is that SCF activates mast cells. The injection of SCF has been shown to cause allergic-like symptoms and the proliferation of mast cells and melanocytes.

FMS-like tyrosine kinase 3 ligand (FLT3L) is an endogenous small molecule that functions as a cytokine and growth factor that increases the number of immune cells (lymphocytes (B cells and T cells)) by activating the hematopoietic progenitors. It acts by binding to and activating FLT3 (CD135) which is found on what (in mice) are called multipotent progenitor (MPP) and common lymphoid progenitor (CLP) cells. It also induces the mobilization of the hematopoietic progenitors and stem cells in vivo which may help the system to kill cancer cells. FLT3L is crucial for steady state plasmacytoid dendritic cell (pDC) and classical dendritic cell (cDC) development. A lack of FLT3L results in low levels of dendritic cells.

In one aspect, contacting the population of CD34⁺ precursor cells includes: (i) contacting the CD34⁺ precursor cells with IL-3, IL-7, IL-15, SCF and FLT3L for about 5-10 days; and (ii) contacting the CD34⁺ precursor cells with IL-7, IL-15, FLT3L and SCF for about at least about 7-21 days. For examples, the population of CD34⁺ precursor cells is cultured with IL-3, IL-7, IL-15, SCF and FLT3L for about 5, 6, 7, 8, 9, 10 or more days, and subsequently cultured with IL-7, IL-15, FLT3L and SCF (i.e., in the absence of IL-3) for at least about 7, 8, 9, 10, 11, 12, 13, 14, 15, 16, 17, 18, 19, 20, 21, or more days.

Contacting the cells with IL-3 has been found to be optional. Therefore, in another aspect, contacting the population of CD34⁺ precursor cells includes: (i) contacting the CD34⁺ precursor cells with IL-7, IL-15, SCF and FLT3L for about 5-10 days; and (ii) contacting the CD34⁺ precursor cells with IL-7, IL-15, FLT3L and SCF for about at least about 7-21 days. For examples, the population of CD34⁺ precursor cells is cultured with IL-7, IL-15, SCF and FLT3L for about 5, 6, 7, 8, 9, 10 or more days, and subsequently cultured with IL-7, IL-15, FLT3L and SCF for at least about 7, 8, 9, 10, 11, 12, 13, 14, 15, 16, 17, 18, 19, 20, 21, or more additional days.

For NK cell differentiation, the HSCs may be switched to an “undefined medium”. As used herein, the term “undefined basal medium” or “undefined medium” is typically meant to refer to media formulations containing human platelet lysate (hPL) or, alternatively, blood serum as a supplement. Such media containing serum or serum-derived ingredients may be commercially available or prepared “in-house” by users. As used herein, the “undefined medium” is referred to as “hPL-containing medium”. As detailed above, such hPL-containing medium can be “basal,” “supplemented” or “complete”.

By “contacting” it is meant that the HSCs are cultured with one or more agent of interest, added in the hPL-containing medium.

In the methods described herein, pluripotent stem cells are differentiated into CD34⁺ hematopoietic precursor cells, which are then differentiated in NK cells. The methods described herein generates intermediate CD34⁺ hematopoietic precursor cells that are different from hematopoietic stem cells (HSCs), which are CD34⁺/CD45⁺. A transient intermediate population of CD34⁺/CD45⁺ cells emerge from the CD34⁺ hematopoietic precursor cells and vanish thereafter to generate fully differentiated NK cells, characterized among other by being CD56⁺ NK cells. In another aspect, the cells are transiently CD34⁺ and CD45⁺.

In one aspect, contacting the adherent culture of PSCs includes one or more agents selected from about 1-10 μM WNT signaling pathway activator, about 10-100 ng/ml BMP, about 50-500 ng/ml VEGF.

The WNT signaling pathway activator is added to the PSC culture at a concentration that ranges from about 1 μM to 10 μM. For example, the PSC are grown in a culture medium that includes about 1, 2, 3, 4, 5, 6, 7, 8, 9, 10 μM or more. In one aspect, the mixture includes about 8 μM WNT signaling pathway activator.

The BMP is added to the PSC culture at a concentration that ranges from about 5 ng/ml to about 50 ng/ml. For example, the PSC are grown in a culture medium that includes about 5, 6, 7, 8, 9, 10, 15, 20, 25, 30, 35, 40, 45, 50 ng/ml or more. In one aspect, the mixture includes about 25 ng/ml BMP.

As further described in the Examples, BMP4 can be prepared by resuspension in various solution. For example, dry BMP4 can be resuspended with PBS/0.01% HAS or in citric acid (as recommended by the manufacturer). PBS/0.01% HAS may reduce the biological activity of BMP4, as compared to its activity when prepared in citric acid. One of skill in the art would easily recognize that a concentration of 5-50 ng/ml of BMP4 could be significantly lowered if the BMP4 is resuspended in citric acid, and therefore has a greater biological activity.

The VEGF is added to the PSC culture at a concentration that ranges from about 50 ng % ml to about 500 ng/ml. For example, the PSC are grown in a culture medium that includes about 50, 75, 100, 125, 150, 175, 200, 225, 250, 275, 300, 325, 350, 375, 400, 425, 450, 475, 500 ng/ml or more. In one aspect, the mixture includes about 200 ng/ml VEGF.

In another aspect, contacting the adherent culture of PSCs includes about 8 μM CHIR99021, about 25 ng/ml BMP4 and/or about 200 ng/ml VEGFA.

In one aspect, the CD34⁺ precursor cells are contacted with about 1-10 ng/ml IL-3, about 4-40 ng/ml IL-7, about 2-20 ng/ml IL-15, about 4-40 ng/ml SCF and/or about 1-20 ng/ml FLT3L. In another aspect, the CD34⁺ precursor cells are contacted with about 4-40 ng/ml IL-7, about 2-20 ng/ml IL-15, about 4-40 ng/ml SCF and/or about 1-20 ng/ml FLT3L.

The IL-3 is added to the PSC culture at a concentration that ranges from about 1 ng/ml to 10 ng/ml. For example, the PSC are grown in a culture medium that includes about 1, 2, 3, 4, 5, 6, 7, 8, 9, 10 ng/ml or more. In one aspect, the mixture includes about 5 ng/ml IL-3.

The IL-7 is added to the PSC culture at a concentration that ranges from about 4 ng/ml to 40 ng/ml. For example, the PSC are grown in a culture medium that includes about 4, 8, 12, 16, 20, 24, 28, 32, 36, 40 μM or more. In one aspect, the mixture includes about 20 ng/ml IL-7.

The IL-15 is added to the PSC culture at a concentration that ranges from about 2 ng/ml to 20 ng/ml. For example, the PSC are grown in a culture medium that includes about 2, 4, 6, 8, 10, 12, 14, 16, 18, 20 ng/ml or more. In one aspect, the mixture includes about 10 ng/ml IL-15.

The SCF is added to the PSC culture at a concentration that ranges from about 4 ng/ml to 40 ng/ml. For example, the PSC are grown in a culture medium that includes about 4, 8, 12, 16, 20, 24, 28, 32, 36, 40 μM or more. In one aspect, the mixture includes about 20 ng/ml SCF.

The FLT3L is added to the PSC culture at a concentration that ranges from about 2 ng/ml to 20 ng/ml. For example, the PSC are grown in a culture medium that includes about 1, 2, 3, 4, 5, 6, 7, 8, 9, 10, 11, 12, 13, 14, 15, 16, 17, 18, 19, 20 ng/ml or more. In one aspect, the mixture includes about 10 ng/ml FLT3L.

In various aspects, the CD34⁺ precursor cells are contacted with about 5 ng/ml IL-3, about 20 ng/ml IL-7, about 10 ng/ml IL-15, about 20 ng/ml SCF and/or about 10 ng/ml FLT3L. In other aspects, the CD34⁺ precursor cells are contacted with about 20 ng/ml IL-7, about 10 ng/ml IL-15, about 20 ng/ml SCF and/or about 10 ng/ml FLT3L.

In one aspect, contacting the adherent culture of PSCs of a) with a WNT signaling pathway activator and a BMP is for about 2-5 days.

In another aspect, the PSCs are subsequently contacted with a VEGF for about 2-5 days.

In one aspect, contacting the population of CD34⁺ precursor cells with IL-3, IL-7, IL-15, SCF and/or FLT3L is for about 5-10 days and contacting the population of CD34⁺ precursor cells with IL-7, IL-15, FLT3L and/or SCF is for at least about 7-21 days. In another aspect, contacting the population of CD34⁺ precursor cells with IL-7, IL-15, SCF and/or FLT3L is for about 5-10 days followed by contacting the population of CD34⁺ precursor cells with IL-7, IL-15, FLT3L and/or SCF is for at least about 7-21 additional days.

In another aspects, the culture of PSCs is an adherent layer of cells. In some aspects, the layer of cells is grown in a two-dimensional culture system or on microcarriers.

In some aspects, the PSCs are cultures on scaffold composed of microcarriers, which are beads or particles. The beads may be microscopic or macroscopic and may further be dimensioned so as to permit penetration into tissues or compacted to form a particular geometry. In some aspects, the framework for the cell cultures comprises particles that, in combination with the cells, form a three-dimensional tissue. The cells attach to the particles and to each other to form a three-dimensional tissue. Beads or microcarriers are typically considered a two-dimensional system or scaffold.

As used herein, a “microcarriers” refers to a particle having size of nanometers to micrometers, where the particles may be any shape or geometry, being irregular, non-spherical, spherical, or ellipsoid. The size of the microcarriers suitable for the purposes herein can be of any size suitable for the particular application. In some embodiments, the size of microcarriers suitable for the three-dimensional tissues may be those administrable by injection. In some embodiments, the microcarriers have a particle size range of at least about 1 μm, at least about 10 μm, at least about 25 μm, at least about 50 μm, at least about 100 μm, at least about 200 μm, at least about 300 μm, at least about 400 μm, at least about 500 μm, at least about 600 μm, at least about 700 μm, at least about 800 μm, at least about 900 μm, at least about 1000 μm.

In some aspects in which the microcarriers are made of biodegradable materials. In some aspects, microcarriers comprising two or more layers of different biodegradable polymers may be used. In some embodiments, at least an outer first layer has biodegradable properties for forming the three-dimensional tissues in culture, while at least a biodegradable inner second layer, with properties different from the first layer, is made to erode when administered into a tissue or organ.

In some aspects, the microcarriers are porous microcarriers. Porous microcarriers refer to microcarriers having interstices through which molecules may diffuse in or out from the microparticle. In other embodiments, the microcarriers are non-porous microcarriers. A nonporous microparticle refers to a microparticle in which molecules of a select size do not diffuse in or out of the microparticle.

Microcarriers for use in the compositions are biocompatible and have low or no toxicity to cells. The microcarriers may comprise various polymers, natural or synthetic, charged (i.e., anionic, or cationic) or uncharged, biodegradable, or nonbiodegradable. The polymers may be homopolymers, random copolymers, block copolymers, graft copolymers, and branched polymers.

In some aspects, the microcarriers comprise non-biodegradable microcarriers. Non-biodegradable microcapsules and microcarriers include, but not limited to, those made of polysulfones, poly (acrylonitrile-co-vinyl chloride), ethylene-vinyl acetate, hydroxyethyl methacrylate-methyl-methacrylate copolymers. These are useful to provide tissue bulking properties or in embodiments where the microcarriers are eliminated by the body.

In some aspects, the microcarriers comprise degradable scaffolds. These include microcarriers made from naturally occurring polymers, non-limiting example of which include, among others, fibrin, casein, serum albumin, collagen, gelatin, lecithin, chitosan, alginate, or poly-amino acids such as poly-lysine. In other aspects, the degradable microcarriers are made of synthetic polymers, non-limiting examples of which include, among others, polylactide (PLA), polyglycolide (PGA), poly (lactide-co-glycolide) (PLGA), poly (caprolactone), polydioxanone trimethylene carbonate, polyhybroxyalkonates (e.g., poly (hydroxybutyrate), poly (ethyl glutamate), poly (DTH iminocarbony (bisphenol A iminocarbonate), poly (ortho ester), and polycyanoacrylates.

In some aspects, the microcarriers comprise hydrogels, which are typically hydrophilic polymer networks filled with water. Hydrogels have the advantage of selective trigger of polymer swelling. Depending on the composition of the polymer network, swelling of the microparticle may be triggered by a variety of stimuli, including pH, ionic strength, thermal, electrical, ultrasound, and enzyme activities. Non-limiting examples of polymers useful in hydrogel compositions include, among others, those formed from polymers of poly (lactide-co-glycolide); poly (N-isopropylacrylamide); poly (methacrylic acid-g-polyethylene glycol); polyacrylic acid and poly (oxypropylene-co-oxyethylene) glycol; and natural compounds such as chrondroitan sulfate, chitosan, gelatin, fibrinogen, or mixtures of synthetic and natural polymers, for example chitosan-poly (ethylene oxide). The polymers may be crosslinked reversibly or irreversibly to form gels adaptable for forming three dimensional tissues.

In exemplary aspects, the microcarriers or beads for use in the present invention are composed wholly or composed partly of dextran.

In other aspects, the PSCs are cultured on a coated surface including a laminin coating.

In various aspects, the CD34⁺ precursor cell is a CD34⁺ endothelial-like precursor cell.

In one aspect, the NK cells are further collected in suspension in a cell culture medium.

The methods described herein allow for the differentiation of NK cells that grow in suspension, from CD34⁺ endothelial-like precursor cell that are adherent. Therefore, during the course of the differentiation, intermediate and transient CD34⁺ CD45⁺ cells emerge in suspension from the CD34⁺ endothelial-like precursor cells and are differentiated into NK cells. After at least a week in culture, the methods described herein yields at least 80% pure or enriched NK cells in suspension. Since the cells are in suspension, they can easily be aspirated and collected in the culture medium.

In one aspect, the NK cells are at least about 80% enriched.

In an additional embodiment, the invention provides a method of inducing NK cell differentiation from PSCs including: a) generating CD34⁺ hemogenic endothelium (HE) cells by: (i) contacting an adherent culture of PSCs with a WNT signaling pathway activator and a BMP for about 3 days; and (ii) contacting the adherent culture of PSCs of i) with a VEGF for about 4 days; thereby generating a population of cells including at least 80% CD34⁺ HE cells; b) contacting the CD34⁺ HE cells of a) with one or more of IL-3, IL-7, IL-15, SCF and/or FLT3L for about 7 days, thereby generating a transient population of cells including at least 80% CD34⁺/CD45⁺ hematopoietic stem cells (HSCs); and c) subsequently contacting the CD34⁺/CD45⁺ HSCs of b) with one or more of IL-7, IL-15, FLT3L and SCF for at least about 7-21 days, thereby inducing NK cell differentiation from PSCs.

In an further embodiment, the invention provides a method of inducing NK cell differentiation from PSCs including: a) generating CD34⁺ hemogenic endothelium (HE) cells by: (i) contacting an adherent culture of PSCs with a WNT signaling pathway activator and a BMP for about 3 days; and (ii) contacting the adherent culture of PSCs of i) with a VEGF for about 4 days; thereby generating a population of cells including at least 80% CD34⁺ HE cells; b) contacting the CD34⁺ HE cells of a) with one or more of IL-7, IL-15, SCF and/or FLT3L for about 7 days, thereby generating a transient population of cells including at least 80% CD34^{+/CD}45⁺ hematopoietic stem cells (HSCs); and c) subsequently contacting the CD34⁺/CD45⁺ HSCs of b) with one or more of IL-7, IL-15, FLT3L and SCF for at least about 7-21 additional days, thereby inducing NK cell differentiation from PSCs.

In one aspect, differentiated NK cells are CD56⁺, NKp30⁺, NKp44⁺, NKp46⁺, NKG2D⁺, NKG2A⁺, KIR2D⁺ and/or CD16⁺.

NK cells can be identified by the presence of CD56 and the absence of CD3 (CD56⁺, CD3⁻). NK cells (belonging to the group of innate lymphoid cells) are one of the three kinds of cells differentiated from the common lymphoid progenitor, the other two being B and T lymphocytes. NK cells are known to differentiate and mature in the bone marrow, lymph nodes, spleen, tonsils, and thymus, where they then enter into the circulation. NK cells differ from natural killer T cells (NKTs) phenotypically, by origin and by respective effector functions; often, NKT cell activity promotes NK cell activity by secreting interferon gamma. In contrast to NKT cells, NK cells do not express T-cell antigen receptors (TCR) or pan T marker CD3 or surface immunoglobulins (Ig) B cell receptors, but they usually express the surface markers CD16 (FcγRIII) and CD57 in humans. The NKp46 cell surface marker constitutes another NK cell marker of preference being expressed in both humans, several strains of mice and in three common monkey species.

NK cells can be classified as CD56^{bright} or CD56^{dim}. CD56^{bright} NK cells are similar to T helper cells in exerting their influence by releasing cytokines. CD56^{bright} NK cells constitute the majority of NK cells, being found in bone marrow, secondary lymphoid tissue, liver, and skin. CD56^{dim} NK cells are primarily found in the peripheral blood and are characterized by their cell killing ability. CD56^{dim} NK cells are always CD16 positive (CD16 is the key mediator of antibody-dependent cellular cytotoxicity (ADCC). CD56^{bright} can transition into CD56^{dim} by acquiring CD16.

In one aspect, the differentiated NK cells are CD56^{bright} or CD56^{dim}.

In various aspects, the differentiated NK cells are cytotoxic NK cells.

In a further embodiment, the invention provides a kit including: a) an HE induction cocktail including: a WNT signaling pathway activator, a BMP and/or a VEGF, b) a NK induction cocktail including IL-3, IL-7, IL-15, SCF and/or FLT3L; and c) instructions for inducing pluripotent stem cell (PSC) differentiation into NK cells.

In one embodiment, the invention provides a kit including: a) an HE induction cocktail including: a WNT signaling pathway activator, a BMP and/or a VEGF, b) a NK induction cocktail including IL-7, IL-15, SCF and/or FLT3L; and c) instructions for inducing pluripotent stem cell (PSC) differentiation into NK cells.

In one aspect, the kit further includes a laminin-coated surface.

In one embodiment, the invention provides a method of generating terminally differentiated hematopoietic cells from PSCs including: a) generating CD34⁺ hematopoietic precursor cells by: (i) contacting an adherent culture of PSCs with a WNT signaling pathway activator and a BMP, wherein the PSCs are grown on a substrate for about 2-5 days; and (ii) contacting the adherent culture of PSCs with a VEGF for about 2-5 days following step (i), wherein the cells produced after step (ii) are at least about 80% enriched for CD34⁺ cells in the total population of cells; thereby generating CD34⁺ precursor cells; and b) contacting the CD34⁺ precursor cells of a) with a mixture of agents to induce differentiation of the CD34⁺ precursor cells into terminally differentiated hematopoietic cells, thereby generating terminally differentiated hematopoietic cells.

In one aspect, the terminally differentiated hematopoietic cell is an immune cell or a red blood cell. In various aspects, the immune cell is selected from the group consisting of macrophage, T cell, and natural killer (NK) cell.

In another embodiment, the invention provides a method of producing a population of CD34⁺ hematopoietic stem cells including a) contacting a culture of pluripotent stem cells (PSCs) with a WNT signaling pathway activator and a bone morphogenetic protein (BMP), wherein the PSCs are grown on a substrate for about 1-8 days; and b) contacting the culture of PSCs with a vascular endothelial growth factor (VEGF) alone or in combination with a WNT signaling pathway activator and/or a BMP for about 1-8 days following step a), wherein the cells produced after step b) are at least about 80% enriched for CD34+ hematopoietic stem cells in the total population of cells, thereby producing a population of CD34+ hematopoietic stem cells.

In one aspect, contacting the adherent culture of PSCs with a WNT signaling pathway activator, a BMP, and/or a VEGF generates CD34+ hemogenic endothelium (HE). In another aspect, the PSCs are human PSCs (hPSCs). In some aspects, the hPSCs are human induced pluripotent stem cells (hiPSCs) or human embryonic stems cells (hESCs).

In a further embodiment, the invention provides a method of generating hematopoietic stem cells from pluripotent stem cells (PSCs) including a) contacting an adherent culture of induced pluripotent stem cells (iPSCs) with a WNT signaling pathway activator and a bone morphogenetic protein (BMP), wherein the PSCs are grown on a substrate for about 2-5 days; and b) contacting the adherent culture of PSCs with a vascular endothelial growth factor (VEGF) for about 2-5 days following step (a), wherein the cells produced after step (b) are at least about 80% enriched for CD34+ cells in the total population of cells; thereby generating CD34+ precursor cells, thereby generating hematopoietic stem cells.

In one aspect, the iPSCs are human iPSCs (hiPSCs).

Presented below are examples discussing methods of inducing the differentiation of PSCs into CD34⁺ hematopoietic precursor cells, contemplated for the discussed applications. The following examples are provided to further illustrate the embodiments of the present invention but are not intended to limit the scope of the invention. While they are typical of those that might be used, other procedures, methodologies, or techniques known to those skilled in the art may alternatively be used.

### EXAMPLES

### Example 1

### Design of Natural Killer Cells Differentiation Protocol

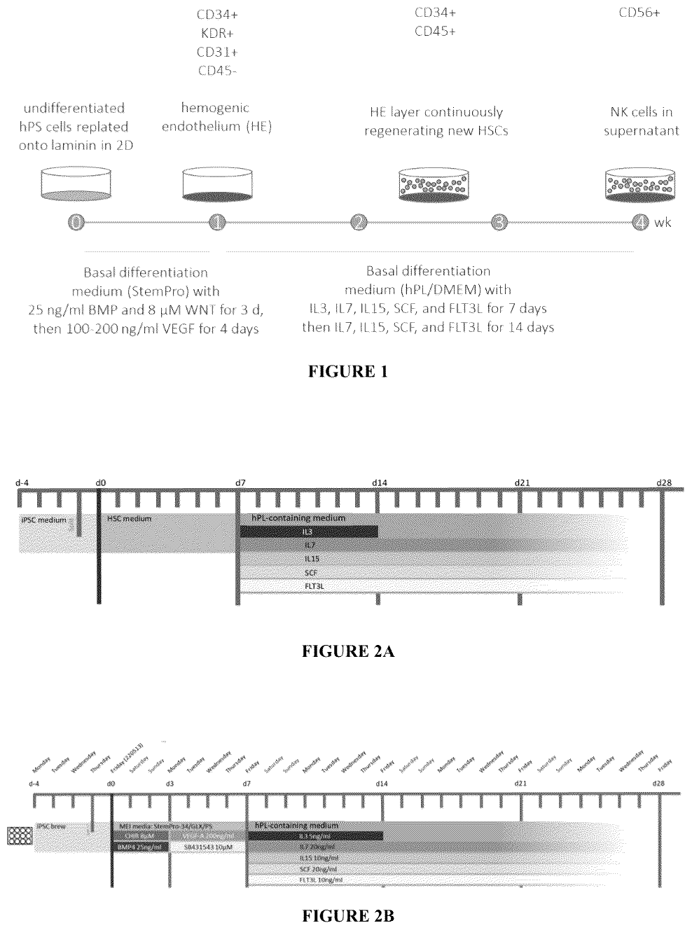

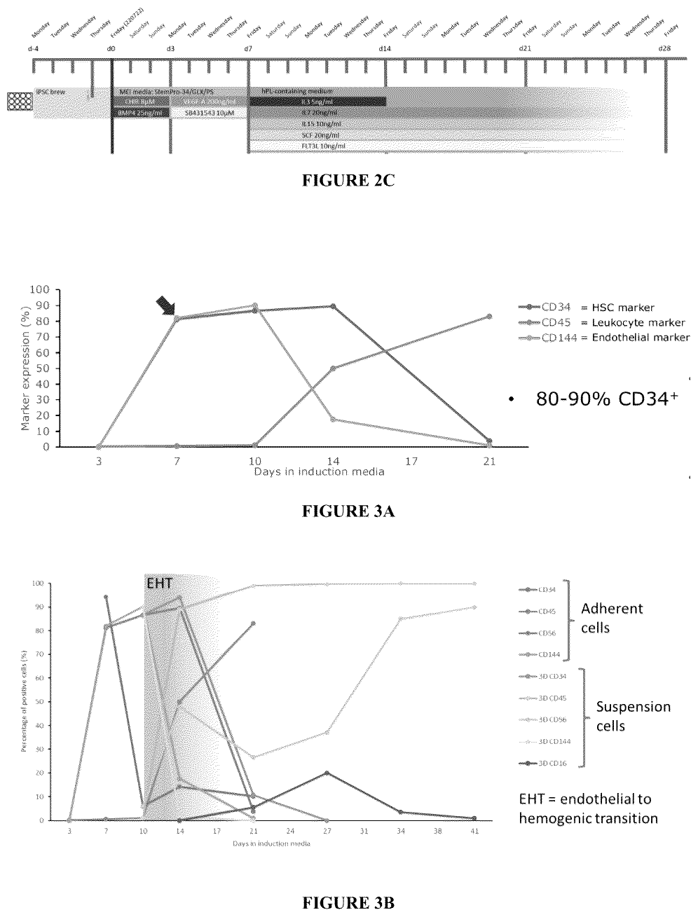

In a stepwise iterative developmental process, a new combination of factors promoting NK cells differentiation at high efficiency has been identified using a simple and GMP-compatible workflow (see FIGS. 1 and 2).

The NK cell differentiation protocol described herein is divided into 2 steps.

The first one aims at differentiating endothelial-like cells from iPSCs, using mesoderm-inducing factors like BMP and WNT, followed by VEGF treatment. The present invention is based on the discovery that endothelial-like precursor cells obtained after a differentiation protocol using BMP and WNT, followed by VEGF treatment display hybrid features of endothelial and hematopoietic precursor cells i.e., hemogenic endothelium (HE)—endothelial-like cells that also form the cells of the hematopoietic system—hematopoietic stem cells at first (marked by CD34 and CD45), then precursors of the myeloid and lymphoid lineages, then terminally differentiated cells like red blood cells, macrophages, T cells, and NK cells. Intriguingly, HE cells indeed co-express endothelial and hematopoietic markers like CD31, CD144 and CD34, respectively.

The protocol described herein uses BMP and or WNT stimulation of adherent iPSCs followed by VEGF treatment to generate HE cells in a defined and controlled manner combined with/followed by treating the cells with one or more of the molecules 1L3, 1L7, IL15, SCF, and/or FLT3L to promote further hematopoietic differentiation and NK cell induction.

HE cells are the common precursors of all hematopoietic cells including all the aforementioned cell types. Therefore, step 1 can also be combined with known induction production procedures for other cell types like T cells. That is, the overall approach may be universal. The optimized procedure generates >90% pure HE cells, then >90% pure HSCs as a key intermediate, then >90% NK cells.

Since the HE cell layer continuously generates new HSCs and because these suspension cells (which bud off from the HE precursors and transition into the supernatant) tend to proliferate, the protocol also generates yields of NK cells.

### Example 2

### Material and Methods

TABLE 1
Material for cell culture

| Material | Reference number/brief instructions |
|---|---|
| iMatrix-511 | Amsbio 892 012 |
| | Store at 2-8° C. Briefly centrifuge vial prior to use. |
| StemMACS iPS-Brew XF Basal | Miltenyi Biotec 130-107-086-500 ml |
| Medium | Store ready to use medium at 2-8° C. |
| StemMACS iPS-Brew XF | Miltenyi Biotec 130-107-087-10 ml |
| Supplement (50×) | Store ready to use below −17° C. |
| Penicillin/Streptomycin (Pen/Str) | ThermoFischer Scientific 15140122 - 100 ml |
| (100×) | Store ready to use below −17° C. |
| StemMACS iPS-Brew XF | Miltenyi Biotec 130-104-368 |
| | Thaw supplement at 37° C., mix well, add to bottle of basal |
| | medium, mix well, aliquot at 45 ml, store at be-low −17° C. For |
| | use, thaw 45 ml aliquot at 37° C., mix well by pipetting (no |
| | inversion). Optionally add 1:100 Pen/Str. Use 37° C. XF medium |
| | to culture hiPSCs. Keep unused remainder at 2-8° C. up to 2 |
| | weeks. |
| Y-27632 (or Y, Rock inhibitor) | R&D TOCRIS 1254/50 |
| | Equilibrate vial to room temperature (RT) and briefly spin |
| | contents. Dissolve entire contents in DMSO to 10 mM (=1000×). |
| | Freeze aliquots in amber 1.5 ml tubes below −17° C. For |
| | use, thaw aliquot at 37° C. or RT and mix by flicking. Aliquots |
| | may be refrozen or stored at 2-8° C. |
| Accutase | Sigma A6964-500 ml |
| | Thaw at 37° C., mix well, and prepare aliquots of 10-12 ml to be |
| | stored below −17° C. For use, thaw aliquot at 37° C. and mix well. |
| | Accutase may be refrozen up to three times. |
| BMP4 | PEPROTECH 120-05ET-10 μg |
| | Equilibrate vial to RT and briefly spin contents. Dissolve entire |
| | contents to 25 μg/ml in 0.1% HSA/PBS. Aliquot in 1.5 ml tubes |
| | and freeze below −17° C. For use, thaw aliquot at 37° C. or RT and |
| | mix by flicking or pipetting. Aliquots may be refrozen up to three |
| | times. Working Concentration: 25 ng/ml. |
| CHIR99021 (or CHIR) | STEMCELL Technologies 72054-10 mg |
| | Equilibrate vial to RT and briefly spin contents. Dissolve entire |
| | contents in DMSO to 8 mM. Freeze aliquots in amber 1.5 ml |
| | tubes below −17° C. For use, thaw aliquot at 37° C. or RT and mix |
| | by flicking. Aliquots may be refrozen. Working Concentration: |
| | 8 μM. |
| VEGF-A | STEMCELL Technologies 78073.1- 100 μg |
| | Equilibrate vial to RT and briefly spin contents. Dissolve entire |
| | contents to 100 μg/ml in 0.1% HSA/PBS. Aliquot in 1.5 ml tubes |
| | and freeze below −17° C. For use, thaw aliquot at 37° C. or RT and |
| | mix by flicking or pipetting. Aliquots may be refrozen up to three |
| | times. Working Concentration: 200 ng/ml. |
| SB431542 (or SB) | Merck 616461- 5 MG |
| | Equilibrate vial to RT and briefly spin contents. Dissolve entire |
| | contents of 5 mg in 650 μl DMSO to 20 mM (=2000×), Aliquot |
| | in amber 1.5 ml tubes and freeze below −17° C. For use, thaw |
| | aliquot at 37° C. or RT and mix by flicking or pipetting. Aliquots |
| | may be refrozen up to three times. Working Concentration: 10 |
| | μM. |
| IL3 | R&D Systems 203-IL-050 |
| | Equilibrate vial to RT and briefly spin contents. Dissolve entire |
| | contents to 100 μg/ml in 0.1% HSA/PBS. Aliquot in 1.5 ml tubes |
| | and freeze below −17° C. For use, thaw aliquot at 37° C. or RT and |
| | mix by flicking or pipetting. Aliquots may be refrozen up to three |
| | times. Working Concentration: 5 ng/ml. |
| IL7 | R&D systems 207-IL-010/CF |
| | Equilibrate vial to RT and briefly spin contents. Dissolve entire |
| | contents to 100 μg/ml in 0.1% HSA/PBS. Aliquot 100 μl in 1.5 |
| | ml tubes and freeze below −17° C. For use, thaw aliquot at 37° C. |
| | or RT and mix by flicking or pipetting. Aliquots may be refrozen |
| | up to three times. Working Concentration: 20 ng/ml |
| IL15 | R&D Systems 247-ILB-025/CF |
| | Equilibrate vial to RT and briefly spin contents. Dissolve entire |
| | contents to 10 μg/ml in 0.1% HSA/PBS. Aliquot in 1.5 ml tubes |
| | and freeze below −17° C. For use, thaw aliquot at 37° C. or RT and |
| | mix by flicking or pipetting. Aliquots may be refrozen up to three |
| | times. Working Concentration: 10 ng/ml |
| SCF | R&D Systems 7466-SC-025/CF |
| | Equilibrate vial to RT and briefly spin contents. Dissolve entire |
| | contents to 20 μg/ml in 0.1% HSA/PBS. Aliquot in 1.5 ml tubes |
| | and freeze below −17° C. For use, thaw aliquot at 37° C. or RT and |
| | mix by flicking or pipetting. Aliquots may be refrozen up to three |
| | times. Working Concentration: 20 ng/ml |
| FLT3L | R&D Systems 308-FKE-050 |
| | Equilibrate vial to RT and briefly spin contents. Dissolve entire |
| | contents to 10 μg/ml in 0.1% HSA/PBS. Aliquot in 1.5 ml tubes |
| | and freeze below −17° C. For use, thaw aliquot at 37° C. or RT and |
| | mix by flicking or pipetting. Aliquots may be refrozen up to three |
| | times. Working Concentration: 10 ng/ml |
| StemPro-34 SEM (1×) | ThermoFischer Scientific 10640-019- 500 ml |
| | Store ready to use medium at 2-8° C. |
| StemPRO-34 Nutrient Supplement | ThermoFischer Scientific 10641-025- 13 ml |
| | Store ready to use below −17° C. |
| GlutaMAX (100×) | ThermoFischer Scientific 35050-038- 100 ml |
| | Store ready to use below −17° C. |
| Penicillin/Streptomycin (Pen/Str, or | ThermoFischer Scientific 15140122 - 100 ml |
| PS) (100×) | Store ready to use below −17° C. |
| Defined medium | ThermoFischer Scientific 10639-011- 500 ml |
| | For preparing 100 ml Mesoderm & Endothelial Induction |
| | Medium (MEI), thaw frozen StemPRO-34 Nutrient Supplement |
| | at room temperature (RT), mix well, add 2.6 ml StemPRO-34 |
| | Nutrient Supplement to 100 ml StemPro-34 basal medium, then |
| | add 1 ml 100× GlutaMAX as well, mix well by pipetting (no |
| | tube inversion). Optionally add 1:100 Pen/Str. Store ready to use |
| | Endothelial Induction Medium at 2-8° C. Use 37° C. Mesoderm & |
| | Endothelial Induction Medium to further differentiate hiPSCs- |
| | derived mesodermal cells. Keep unused remainder medium at 2- |
| | 8° C. up to 2 weeks. |
| hPL-containing basal medium | For 100 ml NK dif. Medium, add 15 ml hPL (15% final), 1 ml |
| | 100× PS, 1 ml GLX 100× and 100 μl AscP (250 mM stock), bring |
| | up to 100 ml using DMEM F12 1:1. Store at 2-8° C., consume |
| | within 2 weeks |
| hPL-containing medium | To the corresponding basal medium, add IL7, IL15, SCF and |
| | FLT3L. Immediately consume freshly made medium. Warm to |
| | at least room temperature before adding to cell culture. Aliquot |
| | the growth factors so that they are consumed within a maximum |
| | of 3 freeze-thaw cycles |

TABLE 2
Antibodies used for Flow Cytometry (all to Store at 2-8° C. or, alternatively,
at −20° C. after addition of 80% (v/v) glycerol to 10% final):

| Antibody | Manufacturer |
|---|---|
| IgG1 VB423 antibody | Miltenyi Biotec 130-127-220 (VioBright 423-labelled) |
| CD56 antibody | Miltenyi Biotec 130-126-299 (VioBright 423-labelled) |
| IgG1 VB515 antibody | Miltenyi Biotec 130-113-457 (VioBright 515-labelled) |
| CD336 (NKp44) antibody | Miltenyi Biotec 130-120-361 (VioBright 515-labelled) |
| CD159a (NKG2A) antibody | Miltenyi Biotec 130-126-900 (VioBright 515-labelled) |
| IgG1 FITC antibody | Miltenyi Biotec 130-113-437 (FITC-labelled) |
| CD16 antibody | Miltenyi Biotec 130-113-392 (FITC-labelled) |
| CD3 antibody | Miltenyi Biotec 130-113-138 (FITC-labelled) |
| IgG1 APC antibody | Miltenyi Biotec 130-113-446 (APC-labelled) |
| CD34 antibody | Miltenyi Biotec 130-120-514 (APC-labelled) |
| KIR2D antibody | Miltenyi Biotec 130-117-479 (APC-labelled) |
| CD314 (NKG2D) antibody | Miltenyi Biotec 130-111-846 (APC-labelled) |
| CD337 (NKp30) antibody | Miltenyi Biotec 130-112-431 (APC-labelled) |
| IgG1 PE antibody | Miltenyi Biotec 130-113-438 (PE-labelled) |
| CD45 antibody | Miltenyi Biotec 130-113-118 (PE-labelled) |
| CD158e/k (KIR3DL1/DL2) | Miltenyi Biotec 130-116-178 (PE-labelled) |
| CD159c (NKG2C) | Miltenyi Biotec 130-119-776 (PE-labelled) |
| CD335 (NKp46) | Miltenyi Biotec 130-112-121 (PE-labelled) |

### Methods:

Maintenance of hiPSs (R26 Line):

hiPSCs were split on Monday mornings and Thursday afternoons at 200,000-250,000 (up to 6 wells) per 6-well, respectively. Cells for experiments were replated on Thursdays using the same cells as for hiPSC maintenance. Differentiation was initiated on Fridays.

### Monday:

Coat 2-4 6-wells for hiPSC maintenance with 3 μl iMatrix-511 per well in 2 ml XF medium with 10 μM Y-27632 (1:1000 Y-27632: 1 μl Y per 1 ml medium) for at least 1 hour at 37° C.
Prewarm Accutase and approximately 10 ml of XF medium. Transfer required volume of Accutase (1 ml per well to be harvested) to separate tube. Add 1:1000 Y-27632 to both solutions and mix. Maintenance wells of hiPSCs should be 50-100% confluent and overall undifferentiated.
Vigorously agitate plate with cells to collect all dead cells in supernatant. Completely soak off medium and wash with 2 ml PBS. Replace by 1 ml prewarmed Accutase containing Y-27632 and transfer to incubator for 10 min. Most cells should come off by gently agitating the plate. If this is not the case, prolong digestion for 2 more min and so forth, until cells lift off virtually by themselves.
Add 3 ml of XF medium containing Y-27632 to 15 ml tube. Flush cells off from each well by pipetting up and down 3-4 times. Transfer cells from all wells to 15 ml tube containing 3 ml XF medium+Y. Centrifuge at 300 g for 3 min. Supernatant should be clear and cells should form a compact pellet.
Soak off supernatant and resuspend in 2 ml of XF medium+Y per harvested 6-well by 3 times pipetting up and down with a 1 ml pipette. Immediately and gently transfer 10 μl to a counting chamber and quantify cell titer.
Plate out 200,000 cells per well into each precoated 6-well. Transfer to incubator and agitate plate slowly describing and infinity symbol inside the incubator.

### Tuesday:

Feed cells with 2.5 ml prewarmed XF medium per well.

### Wednesday:

Feed cells with 3 ml prewarmed XF medium per well.

### Thursday:

Confirm that cells are sub confluent and fully undifferentiated. In the afternoon, coat wells with 6 μl iMatrix-511 and split cells as above except that only 1-2 wells are used, and cells plated at 200,000 cells per well. The remaining cells in suspension are to be plated onto 6-wells precoated with 6 μl iMatrix-511 at 450,000 cells (50,000 cells per cm²) per each well for the experiment. The number of wells may vary depending on experimental design.
a 12-well differentiation, coat the wells with 3 μl iMatrix-511 in 1 ml PBS for 1 h in the incubator. Plate 200,000 cells per well in 1 ml medium including 10 μM ROCK inhibitor.

### Friday:

In the afternoon, feed maintenance wells of hiPSCs with 6 ml XF medium per well over the weekend.
   Differentiation of hiPSCs (R26 Fine) into Hemogenic Endothelium

### Friday:

Confirm even distribution of hiPSCs of the wells to be used for differentiation. Cells should be flat and form loose colonies at around 70-90% confluence.
Prepare and/or pre-warm slightly more than 6 ml StemPro 34 medium per differentiation well. Thaw required aliquots of CHIR99021 and BMP4 at RT for several (2-5) min, then mix by flicking the tubes. Optionally do the same for additional factors to be tested.
Prepare differentiation media for the different conditions in the best way, depending on the experimental design. For example, if all wells shall receive the same amount of CHIR but different concentrations of BMP4, prepare a master mix of stem cell medium with CHIR. Then distribute into individual 15 (or 50) ml tubes and add required amounts of other factor(s) as appropriate. Alternatively, depending on the design of the experiment, the additional factors can directly be added to the wells following replacement of the consumed maintenance by differentiation medium.
Replace old XF medium by 6 ml fresh stem cell medium following vigorous agitation of the differentiation plate(s). Place back into incubator over the weekend.
12-well version: Use 3 ml of stem cell differentiation media per well.

### Monday:

Prepare and/or pre-warm slightly more than 3.5 ml stem cell differentiation medium per differentiation well. Thaw required aliquots of VEGFA and SB431542 at RT for several (2-5) min, then mix by flicking the tubes. Optionally do the same for additional factors to be tested. Replace old stem cell medium by fresh stem cell medium following vigorous agitation of the differentiation plate(s).
12-well version: Use 1 ml per well.

### Tuesday:

Prepare and/or pre-warm slightly more than 3.5 ml stem cell differentiation medium (same as on Monday) per differentiation well. Thaw required aliquots of VEGFA and SB431542 at RT for several (2-5) min, then mix by flicking the tubes. Optionally do the same for additional factors to be tested. Replace old stem cell medium by fresh stem cell medium following vigorous agitation of the differentiation plate(s).
12-well version: Use 1 ml per well.

### Wednesday:

Prepare and/or pre-warm slightly more than 3.5 ml stem cell differentiation medium (same as on Monday) per differentiation well. Thaw required aliquots of VEGFA and SB431542 at RT for several (2-5) min, then mix by flicking the tubes. Optionally do the same for additional factors to be tested. Replace old stem cell medium by fresh stem cell medium following vigorous agitation of the differentiation plate(s).
12-well version: Use 1 ml per well.

### Thursday:

Prepare and/or pre-warm slightly more than 3.5 ml stem cell differentiation medium (same as on Monday) per differentiation well. Thaw required aliquots of VEGFA and SB431542 at RT for several (2-5) min, then mix by flicking the tubes. Optionally do the same for additional factors to be tested. Replace old stem cell medium by fresh stem cell medium following vigorous agitation of the differentiation plate(s).
12-well version: Use 1 ml per well
   Differentiation of Hemogenic Endothelium into NK Cells
On Friday (day 7) aspirate the old stem cell medium and very gently wash each well once with 1 ml PBS. Replace with prewarmed hPL-containing medium+IL3 at a volume of 1 ml per well in a 12 well plate. Add the media very slowly, expect 0-30% of the clusters to detach. Between days 10 and 14, the first suspension cells should appear. Ideal cultures will have d14 yields of 1.5 million cells per well, while the minimum yield at this point should be 0.5 million cells.
On Friday (day 14) perform a ‘spin change’ by pooling the suspension cells (very gently using a p1000 pipette, dripping the old media over the adhesive layer with as little pressure as possible before transferring the suspension cells to a collection tube) then centrifuging at 300 g for 8 mins and resuspending the pellet in new hPL-containing medium (without IL3). Do not allow the adherent cells to dry out and add half the final volume of fresh media (0.5 ml) immediately after collecting the suspension cells. Ideally use 15 ml tubes for centrifugation. Resuspend the cells in half the final plate media volume (6 ml total), then evenly distribute the cells into the wells (0.5 ml each).
On day 17 (Monday), repeat the spin change procedure however now resuspend in 2 ml per well. From day 20 onwards, perform half media changes (HMC) twice a week every 3-4 days. For example, as early as possible on Mondays, and as late as possible on Thursdays to achieve an ideal media change interval average of 3.5 days. For HMC, carefully tilt the plate towards you approximately 30 degrees, then remove 0.9 ml from the surface of each well with a p1000 without disturbing the layer of suspension cells at the bottom. Gently drip in 1 ml fresh hPL-containing medium, avoiding disturbing the sedimented cells.
For cell number tracking during HMCs, measure the exact media volume after discarding 0.9 ml by taking up 0.8 ml with a p1000, and then taking up the remaining volume of the well by slowly turning the pipette until air is sucked in and record this value. Gently mix this volume, resuspending all suspension cells, then take 10 μl for counting (Hemocytometer or diluting in 90 μl PBS and then using the NC-200). Multiply the per milliliter cell count by the measured media volume for an accurate cell count for that well. Be aware that this disturbs the adhesive layer, decreasing NK cell production compared to undisturbed cells.
Do not isolate the suspension cells from the adherent layer until they are ready for expansion (day 28-35, >90% CD56 positive). Cell numbers should slowly decrease from 4E6 on day 40 to 2.5E6 by day 70.

### Flow Cytometry

For performing flow cytometry 1 ml of culture media with cells were spun down at 400 g for 3 mins, then re-suspended in 1 ml PBS and again pelleted at 400 g for 3 mins.
Distribute 0.5 million cells per staining group in 98 μl PBS in 1.5 ml tubes.
Add 2 μl of respective antibody for staining, tap to mix, and incubate for 20 min at RT in the dark.
Washing steps: Add 400 μl of PBS to each tube then centrifuge at 400 g for 2 mins. Discard the supernatant.
Add 300 μl of PBS to each tube and resuspend. Perform flow cytometry. After the detection is completed, analyze the flow cytometry result with analysis software.

### NK Killing Assay:

Decide the ratios of effector and target cells to be tested. For each, keep the total combined cell number around 1 million +/−30%. Plan to have at least 200K target cells in the group with the lowest number, so that after processing, at least 30K are detected by the p3 gate (Cell trace violet positive) during FACS analysis.
Calculate the total number of target cells+10% spare across all test ratios, including a ‘only target cells’ spontaneous cell death control group. Resuspend these cells in 1 ml of PBS. Keep a final negative control group of 1E6 pure target cells that do not get stained to measure autofluorescence levels, but otherwise go through the same processing steps as the other groups.
Add 0.5 μl of cell trace violet (2.5 μM final concentration) and incubate at 37° C. for 15 mins.
Add 5 ml of hPL-containing medium and incubate a further 10 mins to stop the staining reaction.
During these last two steps (25 mins) prepare 1.5 ml tubes with the required number of NK cells for each test ratio at a concentration of 1E6 NK cells per ml in hPL-containing medium.
After stopping the cell trace violet staining of the target cells, pellet them at 300 g for 5 mins and resuspend them in hPL-containing medium at 1E6/ml (e.g., 3E6 planned cells+300K spare cells resuspended in 3.3 ml hPL-containing medium).
Distribute the target cells into the prepared 1.5 ml tubes of NK cells at the calculated ratios.
Briefly spin down the cells with a pulse in the microfuge. Tap to loosen up any pellets that may have formed.
With the lids open, place the tubes in an incubator at 37° C. and 5% CO₂ for 3.5 hours.
Add 1 μl of cell even caspase 3/7 green reagent to each tube (2 μM final), invert+tap to mix. Pulse down again and return the cells to the incubator for another 25 mins.
Discard 600 μl from the top of each tube, being careful not to disturb the sedimented cells at the bottom, bringing the final volume per tube to 400 μl. Add 0.5 μl of Sytox 7-AAD living/dead stain to each tube, mix and let stand for 5 mins. While adding the Sytox, use the p10 pipette to remove any large cell clumps that might block the FACS machine.
After 5 mins incubation, briefly vortex the cells before FACS analysis, drawing up the entire 400 μl.
FACS setup: take a small sample of stained target cells and observe the baseline values at a slow rate while viewing the blue, green, and far-red channels. Adjust the detector sensitivity so that these negative control cells are mainly in the bottom left corner when viewing a green vs. red scatter plot. Make sure that the blue channel is not overexposed (cell trace violet is very bright). When viewing the SSC vs FSC plot, gate all the cells apart from the debris in the very bottom left corner (including dead cells in the upper left area). Exclude doublets in P2. Use the P3 gate to select only violet cells (compared to unstained control). View the P3 gate using red vs green scatter, with the quadrants set to have approximately 97% of the cell in the bottom left quadrant for the target cells alone. 3% of the cells should be spontaneously apoptotic (top left quadrant) or dead (right half) in this group. Use these gates on all other effector/target ratios, setting the stop gate as 30K events in P3.

### Example 3

### Generation and Characterization of Natural Killer Cells Differentiated from Human Pluripotent Stem Cells

A novel approach was developed for differentiating NK cells from iPSCs that allows for higher yields, higher purity and increased simplicity compared to the current gold standard of the spin-embryoid body (EB) formation. This was achieved by replacing the laborious and undefined EB phase with the directed differentiation of iPSCs into hemogenic endothelial cells that further differentiate into hematopoietic stem/progenitor cells, recapitulating the natural transition of cell types during embryonic development. The protocol has been developed with a GMP-compliant process in mind and will allow for off-the-shelf NK cells derived from H-LA-homozygous iPSC banks. This platform will greatly reduce the barrier-of-entry for oncology research groups in transitioning their self-developed CAR receptors from bench to bedside, expanding patient access to powerful immunotherapy.

The objectives of the study were to develop a new method of producing hematopoietic stem/progenitor cells from iPSCs using precise directed differentiation instead of spontaneous differentiation, to further differentiate these intermediate cell types into NK cells at high purity/yield, to characterize the NK cells using widely recognized NK cell surface markers and demonstrate their activity via killing assay, and to establish a GMP compliant, iPSC-based platform with various common NK-related genetic edits ready for the insertion of a tumor-specific CAR receptor.

iPSCs were maintained by weekly passage of 10,000 iPSCs onto Laminin 511-coated 6-well plates. Cells were grown in Miltenyi iPSC brew X F, with cells reaching 70-90% confluence after 7 days of growth. NK differentiation—iPSCs were seeded in a 12-well format one day prior to starting differentiation. From days 0-7, iPSCs are first differentiated into hemogenic endothelium to generate CD34/CD144⁺ cells. From days 7-14, hPL-containing medium including IL3 is applied (as described by Miller & McCullar). From day 14 onwards, the same medium is used without IL3 with half media changes twice per week (see FIGS. 2A-2C, 3A-3E, and 4A-4F).

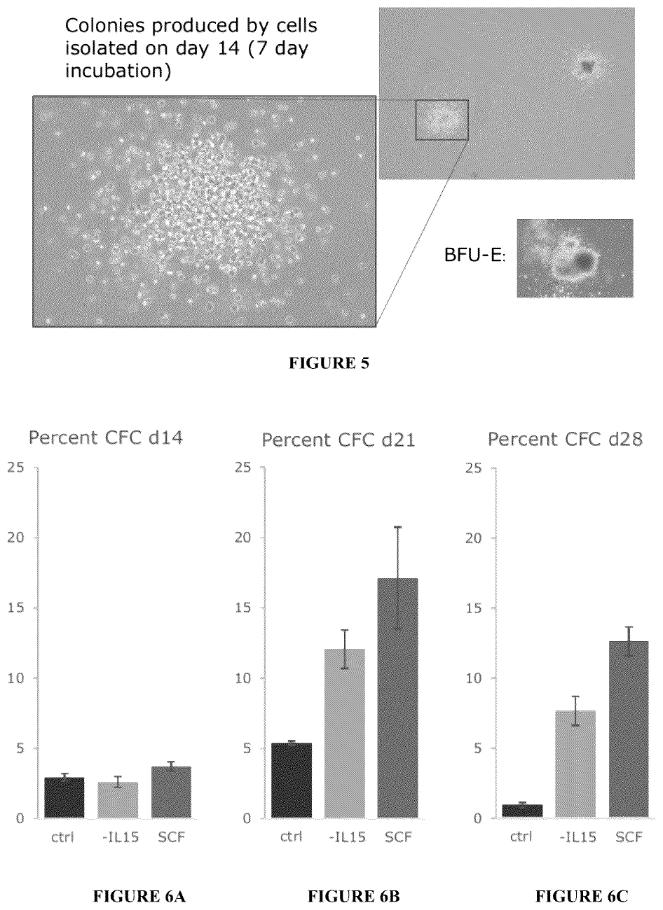

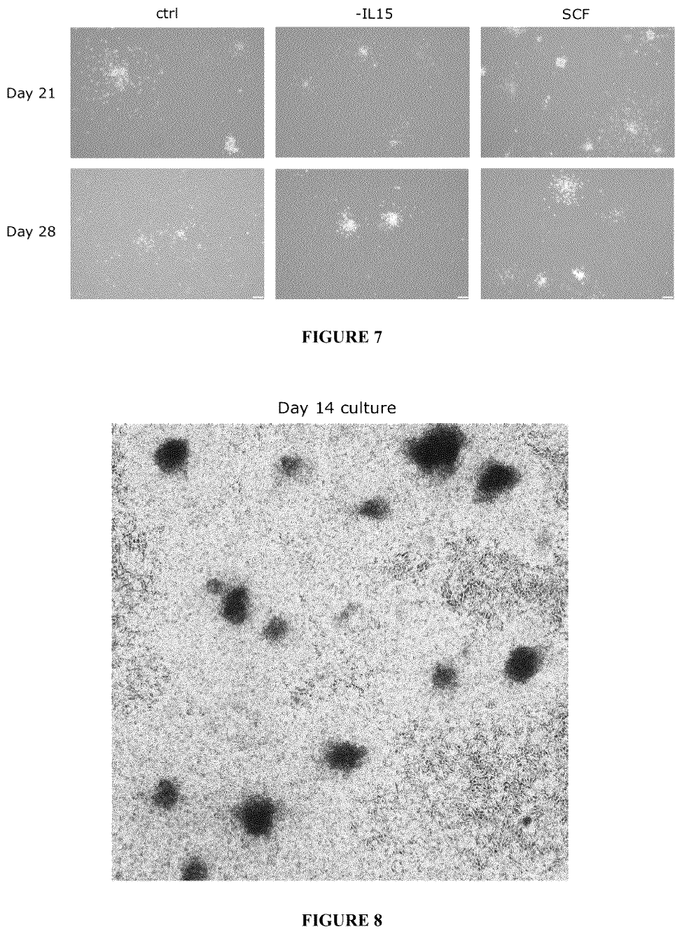

Functional resting of iPSCs-derived hematopoietic stem/progenitor cells was performed. The importance of all the factors during the first week of differentiation of NK cells was assessed using clonogenic assay. 3000 cells were mixed with 2 ml of complete methylcellulose medium (EPO, IL3, GM-CS F, and SCF) and distributed into two wells of a 6-well plate (1 ml=1500 cells per well). After 7 days incubation (37° C., 5% CO2), colonies (defined as clusters of >20 cells) were quantified. Removing factors during the first week of NK differentiation (d7-14) had little effect on colony forming cell (CFC) yield (see FIGS. 6A-6C), however, at later timepoints these conditions increased CFC yield at the expense of target cell differentiation. The vast majority of d14 colonies were of the granulocyte/macrophage variety with rare (burst forming unit erythrocyte) BFU-E colonies (see FIG. 5). Colony forming cells from different differentiation conditions produced smaller colonies over time. Ctrl condition represents complete differentiation medium with all factors, −IL15 represents medium without IL15 and SCF represents medium with only SCF (all had IL3 in the first week) (see FIGS. 7 and 8).

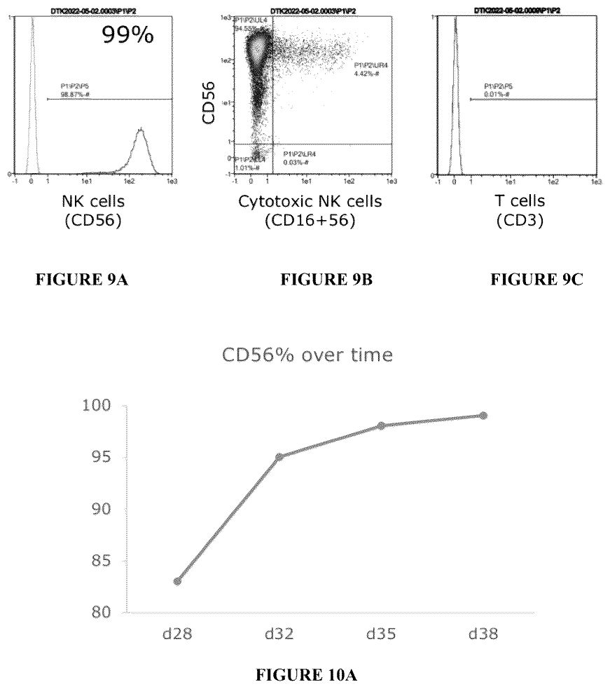

Purity of functional NK cells and long-term culture stability were assessed. Flow Cytometry was performed using a Miltenyi MACS Quant10 flow cytometer. Cells were stained using Miltenyi 1:50 REAfinity FACS antibodies, washed, and then immediately analyzed. Gating: P1: lymphocyte region, P2: doublet exclusion, P3: CD56⁺ cells (NK marker panels only). In each case, 30,000 cells were measured at the final gate. Isotype control antibodies were used as negative controls (see FIGS. 9A-9C).

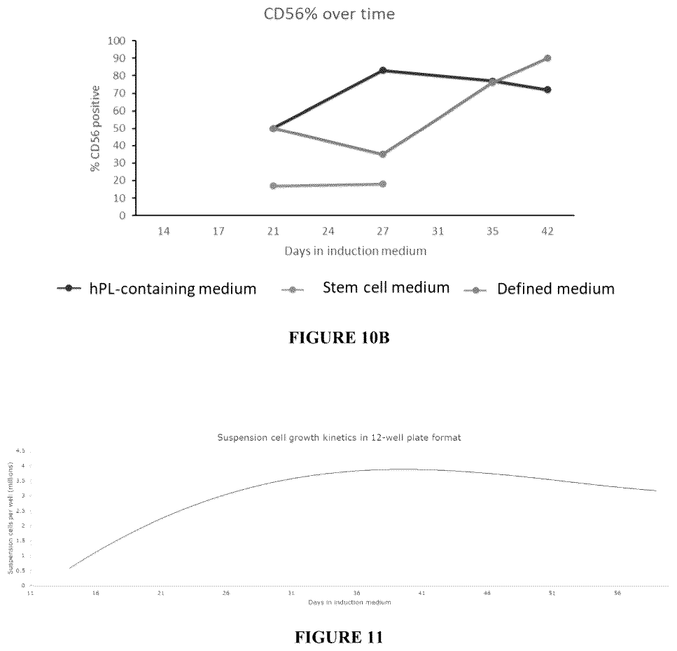

As shown in FIGS. 10A-10B, highly pure NK cells were obtained after 2-3 weeks of HSPC induction, as illustrated by the percent CD56 expression over time. As illustrated in FIGURE JOB, the basal medium used for cell differentiation had an impact on the percent of CD56⁺ cells obtained over the course of the differentiation protocol.

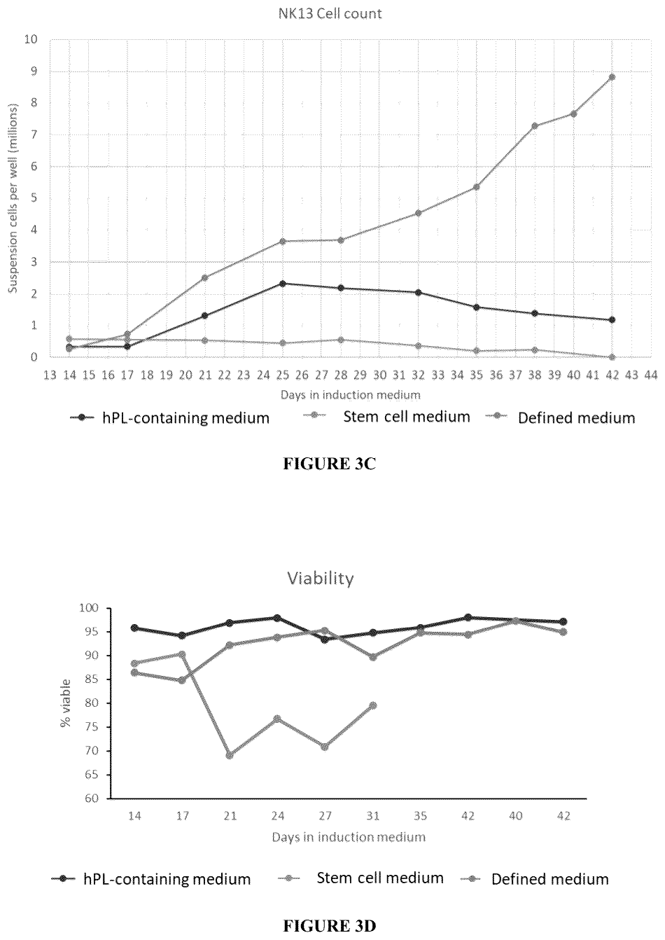

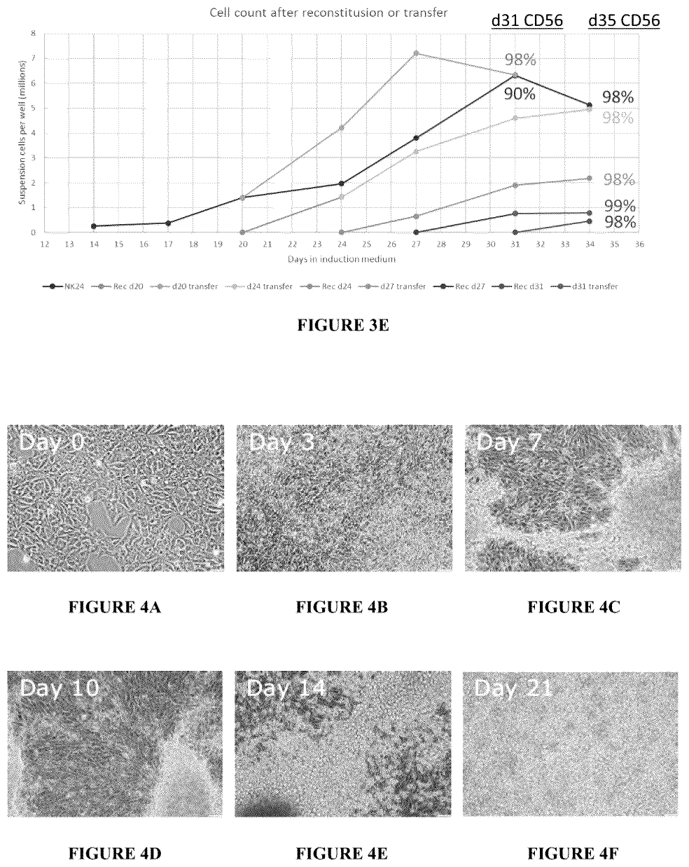

As shown in FIG. 11 NK cells were present and harvested in the culture medium without major decrease in the number of cells obtained for as long as 56 days. Following an exemplary protocol as described in FIG. 2C for example, HE cell layer continuously generated new HSCs and because these suspension cells (which bud off from the HE precursors and transition into the supernatant, they were easily harvested in the cell culture supernatant. As evidenced at FIGS. 3B and 3E, at earlier times, cells in suspension were not all be NK cells (as shows by the absence of CD56 marker), but the proportion of NK cells increased over time, to reach enrichment of at least 80% and up to 98% NK cells. As detailed in FIG. 3E, the timing of cells transfer had an impact of cell reconstitution, and on the number of cells harvested.

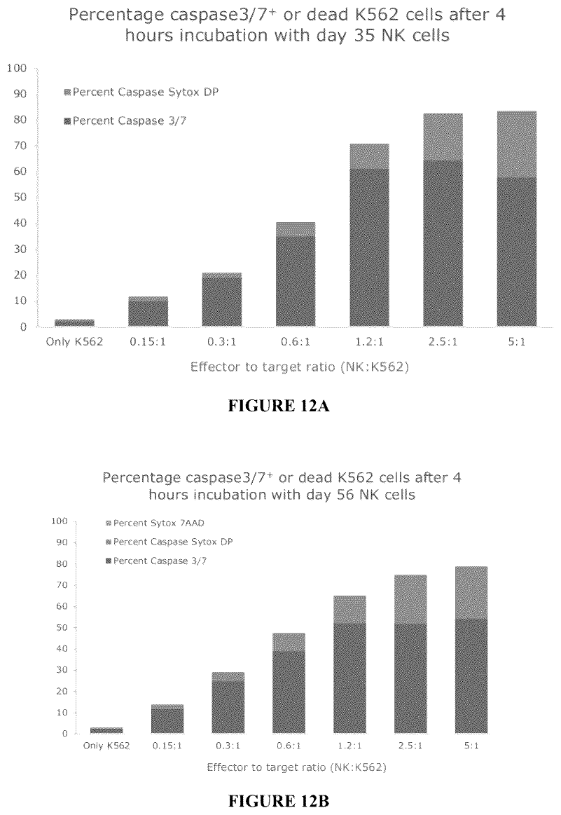

Killing assays were performed at different times during the differentiation protocol. K562 target cells (strain: ACC10) were stained with cell trace violet. The cells were mixed with NK cells at the indicated ratios. They were incubated for 4 hours (37° C., 5% CO₂). 30 mins before the end, cell event caspase 3/7 green was added. 5 mins before the end, Sytox 7AAD living/dead stain was added. The cells were analyzed by flow cytometry. Gating: P1: cell fragment exclusion, P2: doublet exclusion, P3: Cell trace violet positive cells (K562 target cells). Stop gate: 30,000 events in the P3 gate (see FIGS. 12A and 12B). The NK cells produced by this protocol showed robust killing of target K562 cells after 35 days. This killing capacity was unchanged when measured after 56 days of culture.

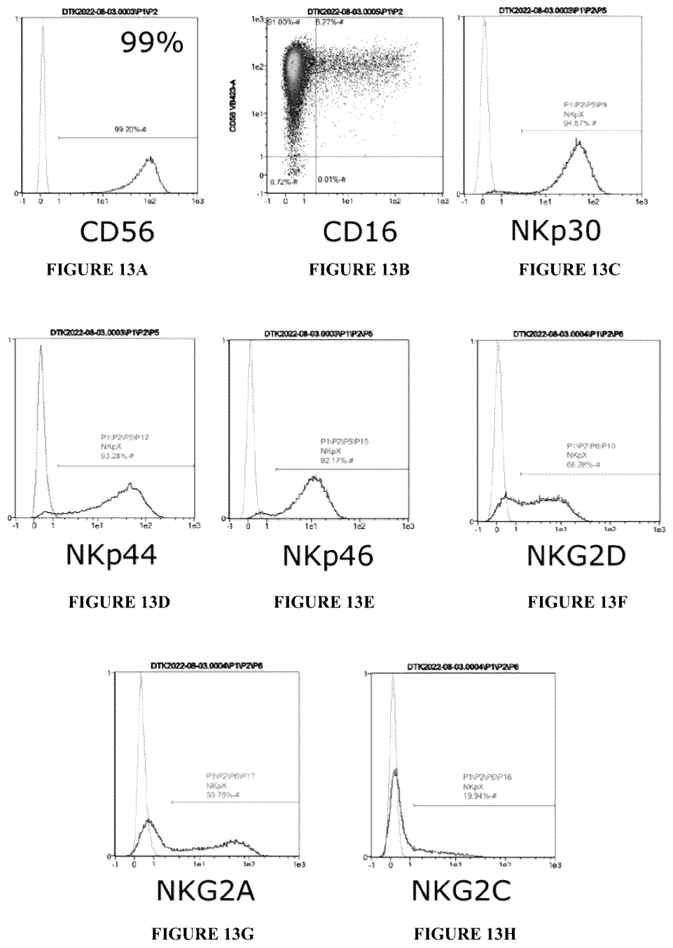

The cells were also further characterized by flow cytometry to assess the expression of different cell surface markers, including CD56, CD16, NKp30, NKp44, NKp46, NKG2D, NKG2A, NKG2C, KIR2D and KIR3D (see FIGS. 13A-13I). NK cells generated via this method are strongly positive for natural cytotoxicity receptors including NKp30, NKp44 and NKp46, as well as the majority being positive for NKG2D. Only around 5% of the NK cells are positive for CD16, suggesting a low capacity for antibody directed cellular cytotoxicity. Additionally, inhibitory receptors of the KIR family were only detected on a small proportion of the cells, possibly increasing the cytotoxicity of these NK cells.

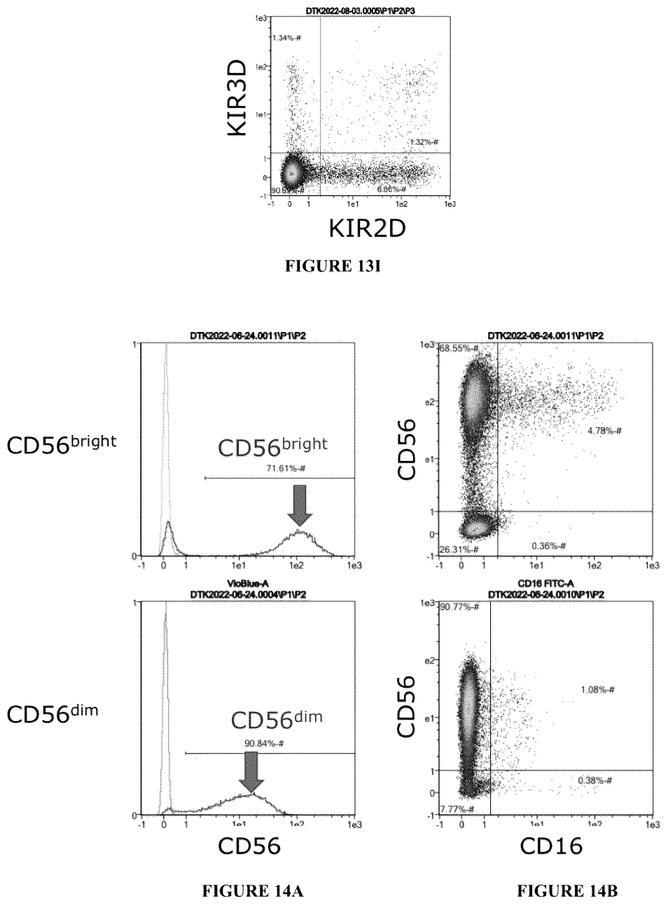

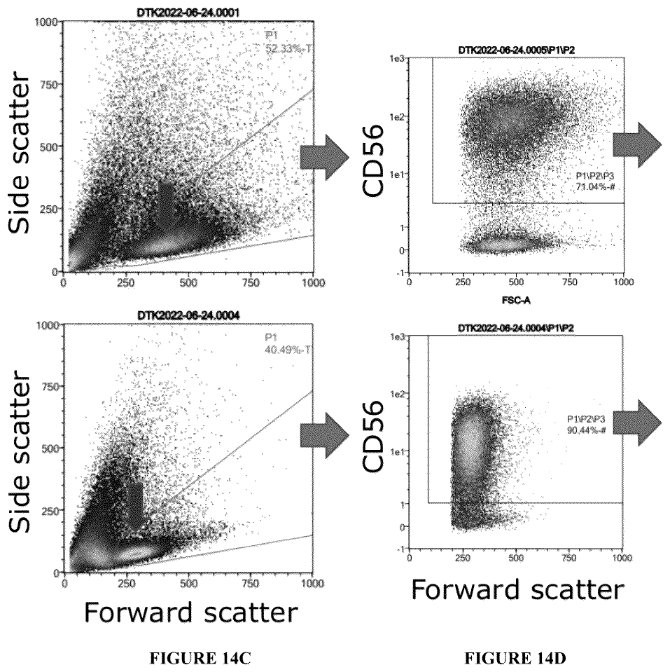

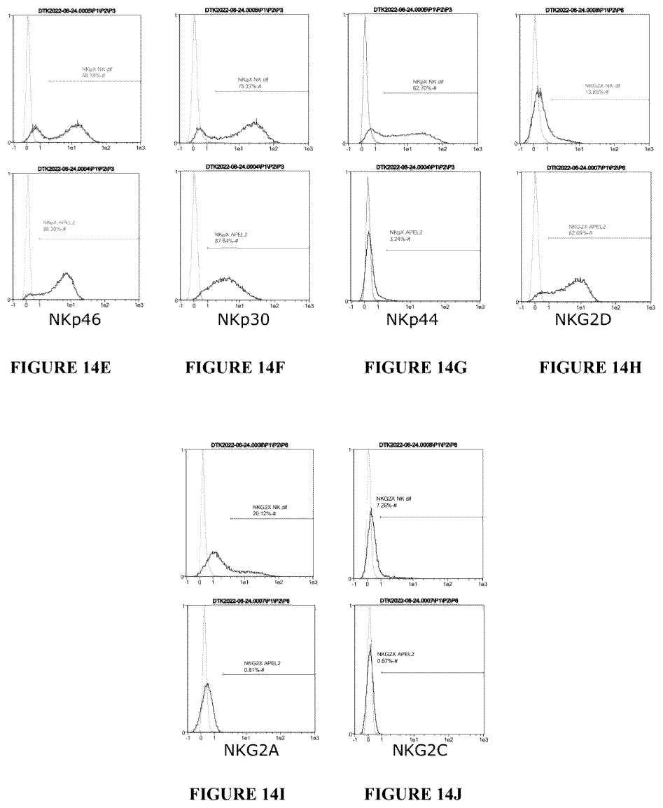

The cells were further analyzed to assess if the differentiation protocol yielded CD56^{bright} and CD56^{dim} NK cells (see FIG. 14A-14J). CD56^{dim} NK cells can be differentiated using completely defined medium and without feeder cells. These cells display a different surface marker profile, with very few being CD16- or NKp44-positive, but with a high number of NKG2D-positive NK cells. The right arrows indicate the cells gated into the next graph, and subsequently the percentage shown refers only to the total cells in the previous gate. In FIG. 14C, the lymphocyte region (p1) is gated and displayed in FIG. 14D, then the CD56 positive cells (p3) are gated and only these are shown in all subsequent histograms. The P2 gate is not shown but filters out approximately 10% of cells which are doublets. In FIGS. 14E-14J, the faint line on the right of each histogram indicates the reading of cells treated with the isotype control antibody (used as a negative control).

To induce the generation of CD56^{bright} and CD56^{dim} NK cells, the hPL-containing medium (DMEM F12+15% hPL) was substituted for a defined serum-free and animal-component-free PSC differentiation medium from day 7 onwards. As illustrated in FIGS. 2C and 3C-3D, respectively, which provide for the experiment design and results including cell counts and viability results. This experiment also tested using a stem cell medium (StemPro34^{T}m) throughout differentiation (instead of only days 1-7), however this did not work, as seen in the suspension cell count results (see FIG. 3D).

A protocol for differentiating iPSCs into HSPCs using precisely defined conditions to yield a large number of CD34+ cells was successfully developed. These cells are capable of producing mostly CFU-GM colonies with a small number of BFU-E colonies when analyzed by a clonogenic assay. These HSPC intermediate cells could be further differentiated into NK cells when exposed to a cocktail of growth factors. After initial optimization of the differentiation conditions, NK cell purity of 99% b was achieved with a total yield of approximately 50 million NK cells per plate after 35 days of culture in a 12-well format. After further optimization, the same yield and purity was achieved after just 28 days (data not shown).

### Example 4

### Generation of T Cells from Isolated iPSC-Derived HSC-Like Cells

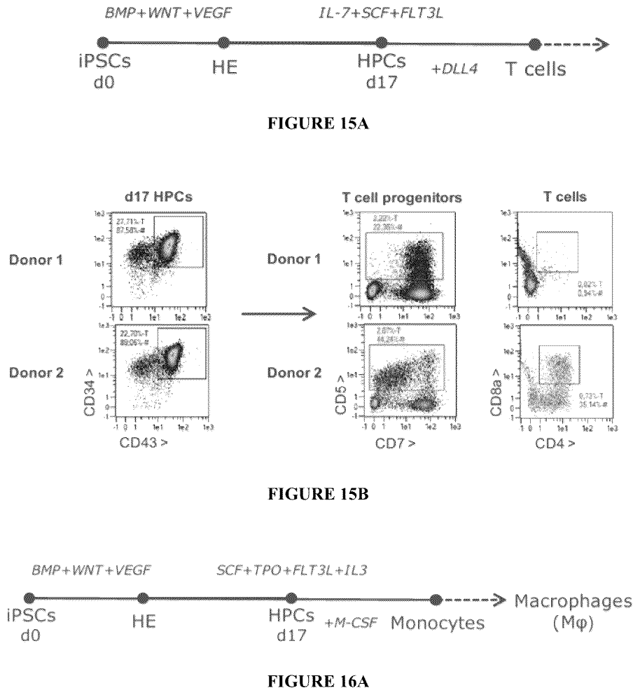

As illustrated in FIG. 15A, the method of the invention can be employed to generate HE and HSC cells as described above, which were then differentiated in T cells using a combination of IL-7, SCF and FLT3L.

As shown in FIG. 15B (left flow cytometry panel), HSC-like hematopoietic precursor cells marked by CD34 and CD43 can be readily obtained from independent iPSC cells. As illustrated in the right panels of FIG. 15B, using the stimulation of the Notch signaling, the HPCs can further be differentiated into T cells progenitors and definitive T cells marked by CD5 and CD7, and CD4 and CD8, respectively. Although there may be iPSC line dependent effect, these data support the concept that the iPSC-derived HSCs are multipotent, and able to give rise to key lymphoid cell types such as T cells.

### Example 5

### Generation of Monocytes/Macrophages from Isolated iPSC-Derived HSQ-Like Cells

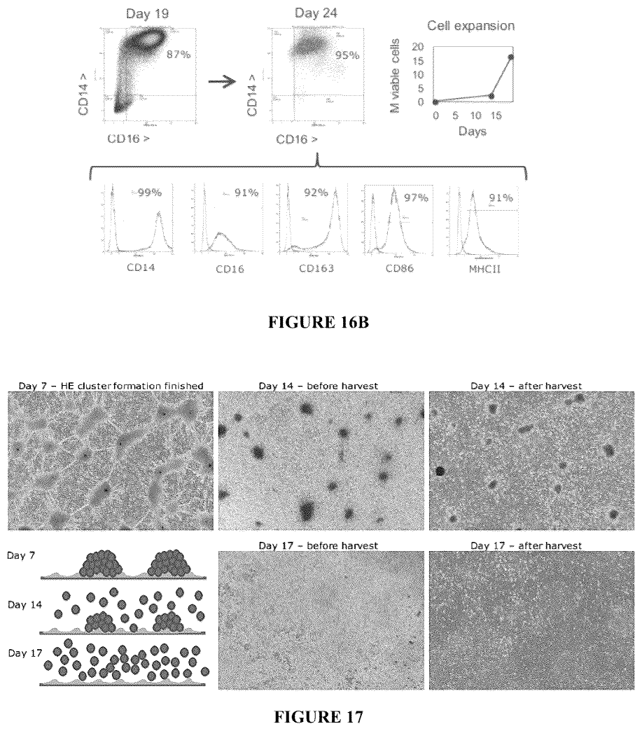

Based on the protocol illustrated in FIG. 16A, which relies on the generation and transfer of HPC intermediate cells to new culture vessels, monocytes/early macrophages were obtained using M-CSF stimulation. The procedure yielded essentially pure monocytes/macrophages marked by CD14, CD16, CD163, CD86 and MHC class II (as illustrated in FIG. 16B).

Differentiation was also accompanied by substantial cell expansion. The data underscores the multipotency of the iPSC-derived HSCs as they can readily be differentiated along the myeloid lineage.

### Example 6

### HSC Production and Differentiation Platform

Using the protocol described in the above examples, an HSC production platform was developed. As illustrated in FIG. 17 (top left), at day 7, in a typical culture HE clusters were visible and their formation complete. They were seen as small white dots on the well surface of the culture vessel. By day 14 of differentiation (see FIG. 17, top middle), where the endothelial-to-hematopoietic transition was underway, HSCs were visible as white halos of dispersing cells surrounding the HE clusters.

Clonogenic assay performed at that point shown multilineage HSC potency. Notably, flow cytometry data analysis showed CD34+, CD90+, CD38− HSC identity (data not shown).

As illustrated in FIG. 17, bottom left panel, the endothelial to hematopoietic transitions (EHT) happened between days 7 and 17 when using an hPL-containing medium. On day 7 (FIG. 17, top left) the hemogenic endothelium (HE) formed clusters on top of an endothelial monolayer. On day 14 (FIG. 17, top middle), the EHT of the clusters was underway, with many suspension cells visible. When removing these suspension cells and replacing the medium, the HE clusters were clearly still visible (FIG. 17, top right). On day 17 (FIG. 17, bottom middle), most of the HE clusters have completely dispersed into the supernatant in the form of an HSC suspension culture. Removing these cells and replacing the medium revealed an endothelial monolayer without clusters (FIG. 17, bottom right).

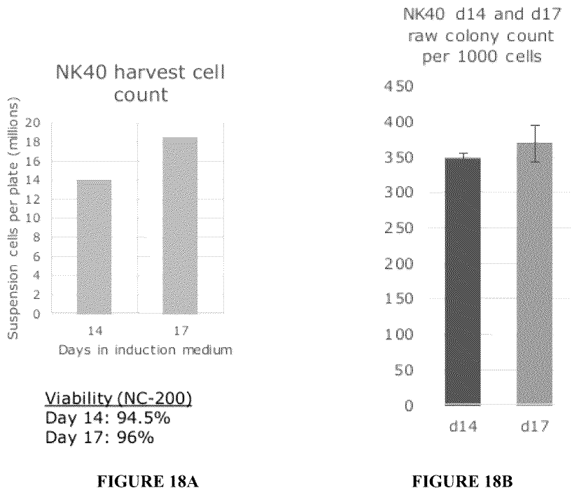

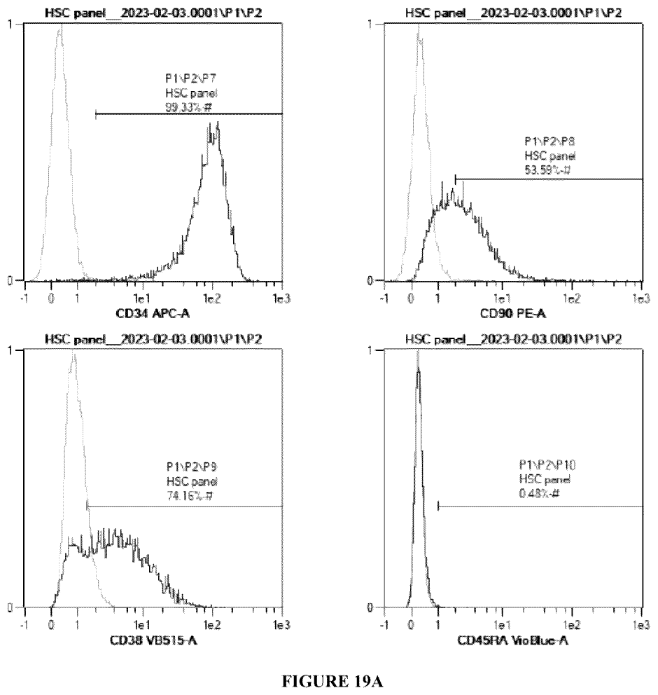

As illustrated in FIG. 18A, when using an hPL-containing medium to generate HSCs, it was observed that approximately half of the EHT was done by day 14, and the remaining half was complete by day 17. Typically, yields between 1 and 3 million HSCs can be expected by day 14 from a single well of a 12-well plate. Clonogenic assay of the day 14 cells after 4 additional days of culture shown that the HSCs generated were capable of forming colonies of white blood cells. Clonogenic assay medium used was methylcellulose containing 1L3, IL6, EPO and SCF.

As shown in FIG. 18B, counting the number of colonies produced from seeding 1000 HSCs on day 14 or 17 revealed that approximately 35% of the cells were capable of producing a colony (defined as >20 cells). The analysis of the colonies classified them as being mostly granulocyte/macrophage (GM-CFU) colony forming units. A low number of red “burst forming unit erythrocyte” (BFU-E) colonies are also present.

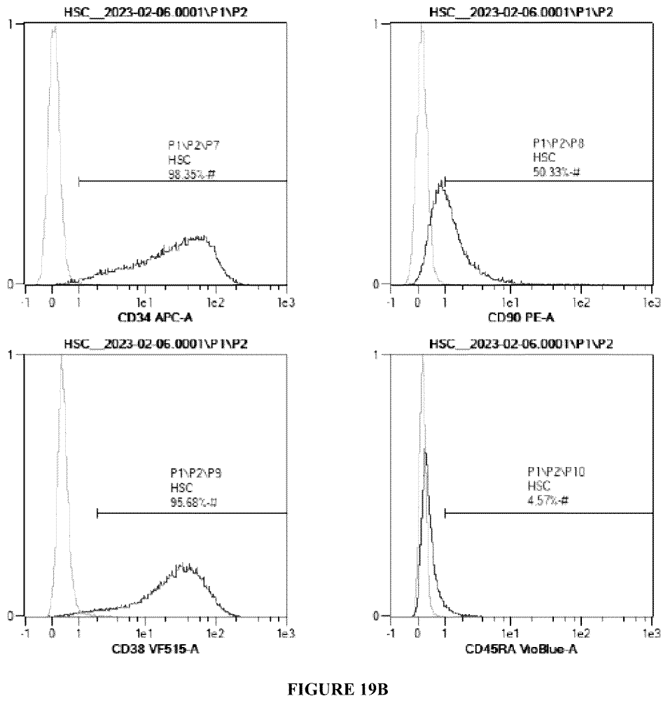

As shown in FIG. 19A, day 14 surface marker analysis revealed a population of CD34+, CD90+, CD38− HSCs via flow cytometry. Day 17 surface marker analysis revealed a small population of CD34+, CD90+, CD38− HSCs via flow cytometry, with a clear trend towards differentiation visible (CD38+) (FIG. 19B).

### Example 7

### Producing HSCs in Various Cell Culture Media

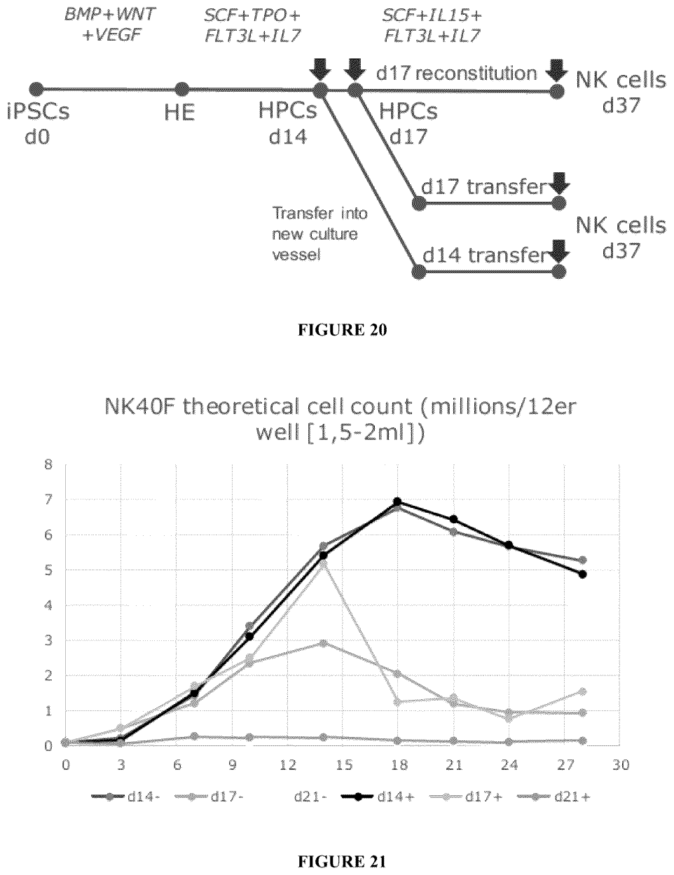

Between days 7 and 17, the hemogenic endothelial clusters undergo an endothelial-to-hematopoietic-transition (EHT) during which the adherent cells disperse into the supernatant to form a suspension cell culture of HSCs. This was originally demonstrated using hPL-containing medium containing human platelet lysate (hPL). It was thus investigated whether the medium had an effect, and it was demonstrated by using fully defined serum-free and animal-component-free PSC differentiation medium, that different types of HSC can be generated with different differentiation abilities and surface marker profiles. The HSCs from four different basal media, were characterized either with supporting cytokines (4F) or without any additional factors (0F), as well as their subsequent ability to differentiate into NK cells. All suspension cells were isolated on day 14 and transferred into new culture vessels for continued differentiation. This was repeated on day 17 with the suspension cells that had arisen since day 14. Finally, the cells that reconstituted the original well after day 17 were also evaluated. On day 38, suspension cells from all three groups were analyzed for NK identity. The protocol is schematically illustrated in FIG. 20, where the arrows indicate the analysis points. Cells with the surface marker profile of: CD34+/CD90+/CD38− were considered HSCs.

On day 14, all conditions (hPL-containing medium, defined medium 1, defined medium 2, and defined medium 3) were capable of producing CD34+/CD90+/CD38− HSCs. The self-made defined medium 2 comprised AscP, VA, Albumin (human), lipids (chemically defined), ITS-X and IMD. Cytokines added to the basal medium included 5 ng/ml IL3, 20 ng/ml TPO, 20 ng/ml SCF, and 10 ng/ml FLT3L. The numbers of CD90+/CD38− cells, representing the total calculated HSCs in single well of a 12-well plate at the time of analysis were as follow:

| | |
|---|---|
| hPL-containing medium (no cytokine): 2,300 cells | hPL-containing medium (all cytokines): 3,600 cells |
| defined medium 1 (no cytokine): 2,500 cells | defined medium 1 (all cytokines): 3,400 cells |
| defined medium 2 (no cytokine): 3,000 cells | defined medium 2 (all cytokines): 5,500 cells |
| defined medium 3 (no cytokine): 10,700 cells | defined medium 3 (all cytokines): 41,500 cells. |

On day 17, all conditions were capable of producing CD34+/CD9+/CD38− HSCs. The numbers of CD90+/CD38− cells, representing the total calculated HSCs in single well of a 12-well plate at the time of analysis were as follow:

| | |
|---|---|
| hPL-containing medium (no cytokine): 7,800 cells | hPL-containing medium (all cytokines): 12,000 cells |
| defined medium 1 (no cytokine): 8,300 cells | defined medium 1 (all cytokines): 39,000 cells |
| defined medium 2 (no cytokine): 81,000 cells | defined medium 2 (all cytokines): 214,000 cells |
| defined medium 3 (no cytokine): 93,000 cells | defined medium 3 (all cytokines): 75,000 cells. |

On day 38 (with reconstitution on day 17), all conditions were capable of producing CD56+ NK cells. The numbers of NK cells, representing the total calculated HSCs in single well of a 12-well plate at the time of analysis were as follow:

| | |
|---|---|
| hPL-containing medium (no cytokine): 1.1M cells | hPL-containing medium (all cytokines): 0.3M cells |
| defined medium 1 (no cytokine): 0.6M cells | defined medium 1 (all cytokines): 2.6M cells |
| defined medium 2 (no cytokine): 1.7M cells | defined medium 2 (all cytokines): 2.7M cells |
| defined medium 3 (no cytokine): 0.2M cells | defined medium 3 (all cytokines): 0.3M cells. |

On day 38 (with transfer on day 14), only DF12/hPL-based cultures were capable of producing CD56+ NK cells. The numbers of NK cells, representing the total calculated HSCs in single well of a 12-well plate at the time of analysis were as follow:

| | |
|---|---|
| hPL-containing medium (no cytokine): 0.5M cells | hPL-containing medium (all cytokines): 0.7M cells |
| defined medium 1 (no cytokine): 0M cells | defined medium 1 (all cytokines): 0M cells |
| defined medium 2 (no cytokine): 0M cells | defined medium 2 (all cytokines): 0M cells |
| defined medium 3 (no cytokine): 0M cells | defined medium 3 (all cytokines): 0M cells. |

On day 38 (with a transfer on day 17), only Nutri T-based cultures were incapable of producing CD56+ NK cells. The numbers of NK cells, representing the total calculated HSCs in single well of a 12-well plate at the time of analysis were as follow:

| | |
|---|---|
| hPL-containing medium (no cytokine): 0.5M cells | hPL-containing medium (all cytokines): 0.3M cells |
| defined medium 1 (no cytokine): 0M cells | defined medium 1 (all cytokines): 0M cells |
| defined medium 2 (no cytokine): 0.4M cells | defined medium 2 (all cytokines): 1.2M cells |
| defined medium 3 (no cytokine): 0.3M cells | defined medium 3 (all cytokines): 0.3M cells. |

### Example 8

### HSCs Cryopreservation

HSCs were cryopreserved on days 14, 17 and 21, and thawed into an hPL-containing medium +/− IL15 (days 0-10 then all +IL15).

It was demonstrated that HSCs generated using an hPL-containing medium could be cryopreserved, then later thawed into culture, and differentiated into NK cells. Half the wells are supplemented with IL15, and half did not for the first 10 days of culture. The other three factors (SCF, FLT3L and 1L7) were supplemented at their normal concentrations in both conditions. As illustrated in FIG. 21, cell counts of the various conditions showed that only cells cryopreserved on day 14 and day 17 cells significantly expand during differentiation.

The flow cytometry data for the differentiating cultures on days 7, 14, 21 and 28 after thaw were analyzed.

On day 7, a large number of CD15-positive cells was observed, suggesting that granulocytes were produced first. A small population of CD14-positive cells was also present, indicating monocytes. The lack of a distinct CD56-positive population meant that no NK cells were present at this point (data not shown).

On day 14, the detection of CD15-positive cells suggested that granulocytes were present. A population of CD14-positive cells indicated the presence of monocytes. A distinct CD56-positive population was visible, indicating that the first NK cells were differentiating in all groups at this point (data not shown).

On day 21, the largest population in most groups was CD14/CD15 double positive. A distinct CD56-positive population was visible indicating that NK cells were differentiating in all groups at this point (data not shown).

On day 28. The majority of the cells were CD56-positive in all groups, confirming that cryopreserved HSCs capable of being thawed into NK differentiating cultures (data not shown).

### REFERENCES

Huang Zhu and Dan S. Kaufman (2019) An improved method to produce clinical scale natural killer cells from human pluripotent stem cells. Methods in Molecular Biology, Vol 2048; Chapter 12, p107-119.
Jeffrey S. Miller and Valarie McCullar (2001) Human natural killer cells with polyclonal lectin and immunoglobulin like receptors develop from single hematopoietic stem cells with preferential expression of NKG2A and KIR2DL2/L3/S2, Blood, Vol 98; 3, p705-713.

Although the invention has been described with reference to the above examples, it will be understood that modifications and variations are encompassed within the spirit and scope of the invention. Accordingly, the invention is limited only by the following claims.

What is claimed is:

1. A method of producing a population of CD34⁺ hematopoietic precursor cells comprising:
a) contacting a culture of pluripotent stem cells (PSCs) with a WNT signaling pathway activator and a bone morphogenetic protein (BMP), wherein the PSCs are grown on a substrate for about 1-8 days; and
b) contacting the culture of PSCs with a vascular endothelial growth factor (VEGF) alone or in combination with a WNT signaling pathway activator and/or a BMP for about 1-8 days following step a),
wherein the cells produced after step b) are at least about 80% enriched for CD34+ cells in the total population of cells,
thereby producing a population of CD34⁺ precursor cells.

2. The method of claim 1, wherein contacting the adherent culture of PSCs with a WNT signaling pathway activator, a BMP, and/or a VEGF generates CD34⁺ hemogenic endothelium (HE).

3. The method of claim 2, further comprising contacting the population of CD34⁺ precursor cells with a mixture of agents to induce differentiation of the CD34⁺ precursor cells to terminally differentiated cells of the hematopoietic lineage.

4. The method of claim 3, wherein the cell of the hematopoietic lineage is a natural killer (NK) cell or another immune cell.

5. The method of claim 1, wherein the culture of PSCs is contacted with a WNT signaling pathway activator and a BMP for about 3 days and with a VEGF for about 4 additional days.

6. The method of claim 1 or 5, wherein the cells treated for about 1 week are CD34⁺, KDR⁺, CD31⁺ and CD45⁻.

7. The method of claim 1 or 5, wherein the CD34⁺ cells are also CD144⁺.

8. A method of producing natural killer (NK) cells comprising:
a) contacting a culture of pluripotent stem cells (PSCs) with a WNT signaling pathway activator and/or a bone morphogenetic protein (BMP), wherein the PSCs are grown on a substrate for about 1-8 days;
b) contacting the culture of PSCs with a vascular endothelial growth factor (VEGF) alone or in combination with a WNT signaling pathway activator and/or a BMP for about 1-8 days following step a), thereby generating a population of CD34⁺ precursor cells, wherein the cells produced after step b) are at least about 80% enriched for CD34⁺ cells in the total population of cells; and
c) contacting the population of CD34⁺ precursor cells with one or more of interleukin-7 (IL-7), IL-15, SCF and FMS-like tyrosine kinase 3 ligand (FLT3L),
thereby producing NK cells.

9. The method of claim 8, wherein contacting the population of CD34⁺ precursor cells of c) comprises:
(i) contacting the CD34⁺ precursor cells with IL-7, IL-15, SCF and FLT3L for about 5-10 days; and
(ii) contacting the CD34⁺ precursor cells with IL-7, IL-15, FLT3L and SCF for about at least 7-21 days.

10. The method of claim 9, wherein the cells are transiently CD34⁺ and CD45⁺.

11. The method of claim 8, wherein contacting the adherent culture of PSCs comprises one or more agents selected from about 1-10 μM WNT signaling pathway activator, about 5-50 ng/ml BMP, about 50-500 ng/ml VEGF.

12. The method of claim 11, wherein the WNT signaling pathway activator a GSK3 inhibitor.

13. The method of claim 12, wherein the GSK3 inhibitor is CHIR99021.

14. The method of claim 11, wherein the BMP is BMP4.

15. The method of claim 11, wherein the VEGF is VEGF-A.

16. The method of claim 11, wherein contacting the adherent culture of PSCs comprises about 8 μM CHIR99021, about 25 ng/ml BMP4 and/or about 200 ng/ml VEGFA.

17. The method of claim 8, wherein c) comprises about 4-40 ng/ml IL-7, about 2-20 ng/ml IL-15, about 4-40 ng/ml SCF and/or about 1-20 ng/ml FLT3L.

18. The method of claim 17, wherein c) comprises about 20 ng/ml IL-7, about 10 ng/ml IL-15, about 20 ng/ml SCF and/or about 10 ng/ml FLT3L.

19. The method of claim 8, wherein contacting the adherent culture of PSCs of a) with a WNT signaling pathway activator and a BMP is for about 2-5 days.

20. The method of claim 8, wherein subsequently contacting the PSCs with a VEGF is for about 2-5 days.

21. The method of claim 11, wherein contacting the population of CD34⁺ precursor cells with IL-7, IL-15, SCF and/or FLT3L is for about 5-10 days followed by contacting the population of CD34⁺ precursor cells with IL-7, IL-15, FLT3L and/or SCF is for at least about 7-21 days.

22. The method of claim 8, wherein the culture of PSCs is an adherent layer of cells.

23. The method of claim 22, wherein the layer of cells is grown in a two-dimensional culture system or on microcarriers.

24. The method of claim 22, wherein the PSCs are cultured on a coated surface comprising a laminin coating.

25. The method of claim 8, further comprising collecting the NK cells in suspension in a cell culture medium.

26. The method of claim 8, wherein the PSCs are human PSCs (hPSCs).

27. The method of claim 26, wherein the hPSCs are human induced pluripotent stem cells (hiPSCs) or human embryonic stems cells (hESCs).

28. The method of claim 8, wherein the CD34⁺ precursor cell is a CD34⁺ endothelial-like precursor cell.

29. The method of claim 8, wherein the NK cells are at least about 80% enriched.

30. A method of inducing natural killer (NK) cell differentiation from pluripotent stem cells (PSCs) comprising:
a) generating CD34⁺ hemogenic endothelium (HE) cells by:
(i) contacting an adherent culture of pluripotent stem cells (PSCs) with a WNT signaling pathway activator and a bone morphogenetic protein (BMP) for about 3 days; and
(ii) contacting the adherent culture of PSCs of i) with a vascular endothelial growth factor (VEGF) for about 4 days:
thereby generating a population of cells comprising at least 80% CD34⁺ HE cells;
b) contacting the CD34⁺ HE cells of a) with one or more of IL-7, IL-15, SCF and/or FLT3L for about 7 days, thereby generating a transient population of cells comprising at least 80% CD34⁺/CD45⁺ hematopoietic stem cells (HSCs); and
c) subsequently contacting the CD34⁺/CD45⁺ HSCs of b) with one or more of IL-7, IL-15, FLT3L and SCF for at least about 7-21 days,
thereby inducing NK cell differentiation from PSCs.

31. The method of claim 30, wherein differentiated NK cells are CD56, NKp30⁺, NKp44⁺, NKp46⁺, NKG2D⁺, NKG2A+, KIR2D⁺ and/or CD16⁺.

32. The method of claim 30, wherein the differentiated NK cells are CD56^{bright} or CD56^{dim}.

33. The method of claim 30, wherein the differentiated NK cells are cytotoxic NK cells.

34. A kit comprising:
a) an hemogenic endothelium (HE) induction cocktail comprising: a WNT signaling pathway activator, a bone morphogenetic protein (BMP) and/or a vascular endothelial growth factor (VEGF)
b) a natural killer (NK) induction cocktail comprising interleukin-7 (IL-7), IL-15, SCF and/or FLT3L; and
c) instructions for inducing pluripotent stem cell (PSC) differentiation into NK cells.

35. The kit of claim 34, further comprising a laminin-coated surface.

36. A method of generating terminally differentiated hematopoietic cells from pluripotent stem cells (PSCs) comprising:
a) generating CD34⁺ hematopoietic precursor cells by:
(i) contacting an adherent culture of pluripotent stem cells (PSCs) with a WNT signaling pathway activator and a bone morphogenetic protein (BMP), wherein the PSCs are grown on a substrate for about 2-5 days; and
(ii) contacting the adherent culture of PSCs with a vascular endothelial growth factor (VEGF) for about 2-5 days following step (i), wherein the cells produced after step (ii) are at least about 80% enriched for CD34⁺ cells in the total population of cells;
thereby generating CD34⁺ precursor cells; and
b) contacting the CD34⁺ precursor cells of a) with a mixture of agents to induce differentiation of the CD34⁺ precursor cells into terminally differentiated hematopoietic cells, thereby generating terminally differentiated hematopoietic cells.

37. The method of claim 36, wherein the terminally differentiated hematopoietic cell is an immune cell or a red blood cell.

38. The method of claim 37, wherein the immune cell is selected from the group consisting of macrophage, T cell, and natural killer (NK) cell.

39. A method of producing a population of CD34⁺ hematopoietic stem cells comprising:
a) contacting a culture of pluripotent stem cells (PSCs) with a WNT signaling pathway activator and a bone morphogenetic protein (BMP), wherein the PSCs are grown on a substrate for about 1-8 days; and
b) contacting the culture of PSCs with a vascular endothelial growth factor (VEGF) alone or in combination with a WNT signaling pathway activator and/or a BMP for about 1-8 days following step a),
wherein the cells produced after step b) are at least about 80% enriched for CD34+ hematopoietic stem cells in the total population of cells,
thereby producing a population of CD34⁺ hematopoietic stem cells.

40. The method of claim 39, wherein contacting the adherent culture of PSCs with a WNT signaling pathway activator, a BMP, and/or a VEGF generates CD34⁺ hemogenic endothelium (HE).

41. The method of claim 39, wherein the PSCs are human PSCs (hPSCs).

42. The method of claim 41, wherein the hPSCs are human induced pluripotent stem cells (hiPSCs) or human embryonic stems cells (hESCs).

43. A method of generating hematopoietic stem cells from pluripotent stem cells (PSCs) comprising:
a) contacting an adherent culture of induced pluripotent stem cells (iPSCs) with a WNT signaling pathway activator and a bone morphogenetic protein (BMP), wherein the PSCs are grown on a substrate for about 2-5 days; and
b) contacting the adherent culture of PSCs with a vascular endothelial growth factor (VEGF) for about 2-5 days following step (a), wherein the cells produced after step (b) are at least about 80% enriched for CD34⁺ cells in the total population of cells; thereby generating CD34⁺ precursor cells,
thereby generating hematopoietic stem cells.

44. The method of claim 43, wherein the iPSCs are human iPSCs (hiPSCs).
